(19) United States
(12) Patent Application Publication
(10) Pub. No.: US 2011/0082195 A1
(43) Pub. Date: Apr. 7, 2011

(54) NEW USE FOR CANNABINOIDS

(75) Inventors: Geoffrey Guy, Dorset (GB); Stephen Wright, Salisbury (GB); Michael Anthony Cawthorne, Buckingham (GB); Saoirse O'Sullivan, Derby (GB)

(73) Assignee: GW PHARMA LIMITED, SALISBURY (GB)

(21) Appl. No.: 12/863,842

(22) PCT Filed: Jan. 21, 2009
(86) PCT No.: PCT/GB09/00159

§ 371 Date: Dec. 6, 2010

(51) Int. Cl. A61K 31/352; A61K 31/045; A61P 3/04; A61P 3/10; A61P 3/00; A61P 9/00

(52) U.S. Cl. 514/454; 514/729

(57) ABSTRACT

The present invention relates to the use of CBD alone or in combination with another cannabinoid, in the manufacture of a pharmaceutical or neutraceutical formulation for use in controlling cholesterol levels in a subject. It also relates to the use of THCV alone or in combination with another cannabinoid, in the manufacture of a pharmaceutical or neutraceutical formulation for use in increasing energy expenditure in a subject. Furthermore the CBD alone or in combination with another cannabinoid or the THCV alone or in combination with another cannabinoid are used as part of a regime to manage or treat type I or II diabetes, obesity, dyslipidaemia, related metabolic disorders and cardiovascular disease.

### FIELD OF THE INVENTION

The present invention relates to the use of CBD alone or in combination with another cannabinoid, in the manufacture of a pharmaceutical or neutraceutical formulation for use in controlling cholesterol levels in a subject. It also relates to the use of THCV alone or in combination with another cannabinoid, in the manufacture of a pharmaceutical or neutraceutical formulation for use in increasing energy expenditure in a subject. Furthermore the CBD alone or in combination with another cannabinoid or the THCV alone or in combination with another cannabinoid are used as part of a regime to manage or treat type I or II diabetes, obesity, dyslipidaemia, related metabolic disorders and cardiovascular disease.

### BACKGROUND DESCRIPTION

Metabolic disorders affect millions of sufferers worldwide and as such cause an increasingly negative impact upon the health of society as a whole.

Diabetes mellitus is a disease of blood sugar (glucose) metabolism. The level of glucose in the blood is normally controlled by the hormone insulin. An increase in blood glucose levels following dietary intake of sugar stimulates the pancreas to produce insulin. The insulin binds to muscle, fat and liver cells and stimulates them to actively take in glucose, as such reducing levels in the bloodstream. Insulin also reduces glucose production by the liver.

Diabetes causes unusually high levels of sugar in the blood. Diabetes is identified based on blood glucose levels measured following a fasting plasma glucose test or an oral glucose tolerance test. High blood sugar may itself cause symptoms, and over a longer period causes damage to the eyes, kidneys, and nerves. This leads to a range of serious complications such as blindness, kidney failure, cardiovascular disease, foot ulcers and gangrene, which may necessitate amputation.

There are two main forms of diabetes:
Type I: Insulin dependent (also called juvenile onset) diabetes; and
Type II: Non-insulin dependent (also called adult onset) diabetes

Individuals with type I diabetes have an autoimmune reaction that destroys the pancreatic beta-cells that produce insulin, so that there is insufficient insulin present in the body. This form of diabetes typically develops before the age of 40, is treated by daily injections of insulin, combined with controlled dietary intake.

The causes of type I diabetes are environmental and genetic in nature, although it is unclear quite what the environmental factors are.

In type II diabetes, insulin is produced by the body, but the cells fail to respond to the insulin and do not take in enough glucose from the blood. This form of diabetes is sometimes referred to as “insulin-resistant” diabetes.

Type II diabetes is often successfully managed by a controlled diet, but in some cases drugs or insulin injections are also required. Patients suffering from type II diabetes initially produce sufficient insulin but because they continue to have high blood sugar levels, the pancreas gradually fails to respond and production of insulin decreases. When this occurs the patient requires treatment with high dose injections of insulin.

The incidence of type II diabetes in the UK and other developed nations is increasing and this is linked to the increasing incidence of obesity in these countries.

The current prevalence of type II diabetes in the UK is around 1 million, whereas prevalence of type I diabetes is around 400,000.

The world-wide prevalence of type II diabetes is estimated to reach 215 million by 2010.

Type II diabetes typically occurs after the age of 40, but there is an increasing trend towards early onset disease among obese teenagers and young adults.

Obesity is defined as having a body mass index (BMI) of 30 or greater. A normal BMI is considered to be in the range of between 18 and 24.9.

It is estimated that in the UK over half the women and about two-thirds of men have a BMI greater than 25 making them either overweight or obese.

Being overweight or obese is known to increase the risk of other diseases including type II diabetes, heart disease, high blood pressure and osteoarthritis.

There are many metabolic disorders related to diabetes and obesity and these are often referred to as “Metabolic Syndrome”. Metabolic syndrome is also known as Insulin Resistance Syndrome or Syndrome X, and these terms refer to a cluster of disorders which commonly occur together in a patient.

The disorders which occur include the following: high or elevated blood pressure; abdominal obesity, where fat has a tendency to be laid down around the abdomen; insulin resistant diabetes or glucose intolerance, cardiovascular disease; atherogenic dyslipidemia, where high triglycerides, low HDL cholesterol and high LDL cholesterol lead to a build-up of plaque in the artery walls; pro-inflammatory state, such as for example elevated C-reactive protein in the blood; and pro-thrombic state, where there is a high fibrinogen or plasminogen activator inhibitor in the blood.

People with metabolic syndrome are known to be at an increased risk of coronary heart disease and other diseases related to the build-up of plaque in the artery walls. Such diseases include stroke and peripheral vascular disease. People with metabolic syndrome are also at an increased risk of suffering from type II diabetes.

The incidence of metabolic syndrome is becoming increasingly common and at the current time it is estimated that over 50 million Americans are sufferers of metabolic syndrome.

The underlying risk factors for metabolic syndrome seems to be abdominal obesity and insulin resistance or type II diabetes, where the body cannot use insulin efficiently.

Other general risk factors include physical inactivity, ageing, hormonal imbalances and a genetic predisposition. Acquired factors such as excess body fat can elicit metabolic disorders such as insulin resistance.

Metabolic syndrome is often diagnosed where individuals present with three or more of the following criteria: elevated waist circumference (equal to or greater than 102cm in males and equal to or greater than 88 cm in females); elevated triglycerides (equal to or greater than 150 mg/dL); reduced HDL “good” cholesterol (less than 40 mg/dL in males and less than 50 mg/dL in females; elevated blood pressure (equal to or greater than 135/85 mm Hg; elevated fasting glucose (equal to or greater than 100 mg/dL).

The primary goal for the clinical management of metabolic syndrome is to reduce the risk of cardiovascular disease and type II diabetes. The risks of these diseases are highly diminished by reducing LDL cholesterol, reducing blood pressure, and reducing blood glucose levels. In addition increasing the level of HDL cholesterol lessens the risk of metabolic syndrome.

At the current time there are no available treatments that are able to reduce or treat metabolic syndrome. As such most of the clinical management of the disease is through lifestyle management.

The use of cannabis as a medicine has long been known and during the 19^{th} Century preparations of cannabis were recommended as a hypnotic sedative which were useful for the treatment of hysteria, delirium, epilepsy, nervous insomnia, migraine, pain and dysmenorrhoea.

Cannabinoids are a group of chemicals known to activate cannabinoid receptors in cells. These chemicals, which are found in cannabis plants, are also produced endogenously in humans and other animals, and are termed endocannabinoids. Synthetic cannabinoids are manmade chemicals with the same structure as plant cannabinoids or endocannabinoids.

The applicant has found in their co-pending application WO2006/054057 that the cannabinoid tetrahydrocannabivarin (THCV) acts as a neutral antagonist at the CB₁ and CB₂ cannabinoid receptor. This has implications for the use of this cannabinoid in the treatment of diseases which are known to be associated with agonism of the CB₁ receptor. These diseases and conditions include the following: obesity; schizophrenia; epilepsy or cognitive disorders such as Alzheimer's disease; bone disorders; bulimia; obesity associated with type II diabetes (non-insulin dependant diabetes); and in the treatment of drug, alcohol or nicotine abuse or dependency.

More recently the applicants have described in their co-pending application GB2438682 the cannabinoid receptor antagonist properties of the cannabinoid cannabidiol (CBD). The cannabinoid CBD acts as an inverse agonist of the CB₁. and CB₂ cannabinoid receptors.

Additionally the application WO 05/077348 describes the use of cannabidiol in the prevention or treatment of diabetes and/or insulitis.

The applicants have found by direct experiment that certain cannabinoids are able to increase energy expenditure; reduce the total cholesterol levels and increase the HDL cholesterol levels. In addition these data from various models of diabetes show desirable effects on plasma insulin, leptin and adiponectin levels. These hormones are of particular relevance to the development and treatment of diabetes, especially in obese individuals. As such these cannabinoids may be very useful for use in the treatment of diabetes, obesity and related metabolic disorders.

The cannabinoid THCV is a classical plant cannabinoid, which is structurally related to THC, in that instead of the 3-pentyl side chain of THC, the THCV molecule has a 3-propyl side chain. The cannabinoid CBD is again another classical plant cannabinoid, which is known to be non-psychoactive. CBD has previously been shown to be useful in the treatment of inflammation, nausea and anxiety.

The applicant has previously proposed formulations of the two cannabinoids THCV and CBD, as has been described in the applicants co-pending application GB0713175.8 (unpublished). It is thought that the combination of the THCV and CBD should provide a better treatment option due to the difference in the ways the two cannabinoids have an effect at the cannabinoid receptors. In this application a ratioed mix of: (i) one or more compounds that acts as an inverse agonist of the CB₁ and/or CB₂ receptor; and (ii) one or more compounds that acts as a neutral antagonist of the CB₁ and/or CB₂ receptor is disclosed.

THCV is thought to act directly on the cannabinoid receptors and bind to cause a neutral antagonist effect. This means that the receptor itself is blocked to binding with an agonist such as an endocannabinoid; however the background tone of the receptor remains unaffected. When THCV is provided as a pharmaceutical formulation alone the unaffected background tone means that some of the diseases and conditions that antagonism is useful to treat may not be fully alleviated as the background tone may still cause an effect on the body.

Conversely, CBD is thought to act as an inverse agonist, which means that the background tone of the receptor is switched off. However, CBD is thought to bind at a site distinct from the cannabinoid receptors themselves and as such may allow an agonist to bind with the receptor.

In another of the applicant's co-pending applications GB0800390.7 (unpublished), the applicant describes the use of one or more cannabinoids in combination with anti-psychotic medications. The side-effects experienced by many users of anti-psychotic medications include diabetes and other related metabolic disorders. The data relating to how the cannabinoids are able to prevent or treat some of these side effects has led the applicant to the belief that one or more cannabinoids may be useful in combination with the anti-psychotic medication to ameliorate the metabolic related side-effects.

Drugs to that are used to treat obesity can be divided into three groups: those that reduce food intake; those that alter metabolism; and those that increase thermogenesis. Monoamines acting on noradrenergic receptors, serotonin receptors, dopamine receptors, and histamine receptors can reduce food intake. A number of peptides also affect food intake. The noradrenergic drugs phentermine, diethylpropion, mazindol, benzphetamine, and phendimetrazine are approved only for short-term use. Sibutramine, a norepinephrine-serotonin reuptake inhibitor, is approved for long-term use. Orlistat inhibits pancreatic lipase and can block 30% of the triacylglycerol hydrolysis in subjects eating a 30% fat diet. The only thermogenic drug combination that has been tested is ephedrine and caffeine, but this treatment has not been approved by regulatory agencies. In clinical trials other drugs that may modulate peptide-feeding systems are being developed. The drug rimonabant elicits its main effect by reduction of appetite.

### SUMMARY OF THE INVENTION

According to a first aspect of the present invention there is provided the use of CBD alone or in combination with another cannabinoid, in the manufacture of a pharmaceutical or neutraceutical formulation for use in controlling cholesterol levels in a subject.

Preferably the pharmaceutical or neutraceutical formulation is for use in reducing total plasma cholesterol.

Preferably the pharmaceutical or neutraceutical formulation is for use in increasing the percentage of HDL cholesterol relative to total cholesterol.

More preferably the other cannabinoid is THCV.

References to THCV and CBD, THCV- and CBD-type compounds or derivatives thereof, particularly with regard to therapeutic use, will be understood to also encompass pharmaceutically acceptable salts of such compounds. The term “pharmaceutically acceptable salts” refers to salts or esters prepared from pharmaceutically acceptable non-toxic bases or acids, including inorganic bases or acids and organic bases or acids, as would be well known to persons skilled in the art. Many suitable inorganic and organic bases are known in the art.

The scope of the invention also extends to derivatives of THCV or CBD that retain the desired activity. Derivatives that retain substantially the same activity as the starting material, or more preferably exhibit improved activity, may be produced according to standard principles of medicinal chemistry, which are well known in the art. Such derivatives may exhibit a lesser degree of activity than the starting material, so long as they retain sufficient activity to be therapeutically effective. Derivatives may exhibit improvements in other properties that are desirable in pharmaceutically active agents such as, for example, improved solubility, reduced toxicity, enhanced uptake.

Preferably the CBD is in the form of a cannabinoid-containing plant extract derived from at least one cannabis plant.

In one embodiment the cannabinoid-containing plant extract from at least one cannabis plant is produced by extraction with supercritical or subcritical CO₂.

Alternatively the cannabinoid-containing plant extract from at least one cannabis plant is produced by contacting plant material with a heated gas at a temperature which is greater than 100° C., sufficient to volatilise one or more of the cannabinoids in the plant material to form a vapour, and condensing the vapour to form an extract.

Preferably the cannabinoid-containing plant extract from at least one cannabis plant is a botanical drug substance.

More preferably the cannabinoid-containing plant extract from at least one cannabis plant comprises all or some of the naturally occurring cannabinoids present in the plant.

More preferably all or a significant amount of any THC occurring in the cannabis-containing plant extract has been removed.

Alternatively the CBD or any other cannabinoid is/are in a substantially pure or isolated form.

Alternatively the CBD or any other cannabinoid is/are in a synthetic form.

Preferably the CBD is present in a dose effective to bring about a reduction in total plasma cholesterol.

Preferably the effective dose of CBD is between 0.1 mg/kg and 5.0 mg/kg.

More preferably, when THCV is present it is present in an amount of between 0.3 mg/kg and 30.0 mg/kg.

Preferably the cholesterol levels are controlled as part of a regime to manage or treat type I or type II diabetes, obesity, dyslipidaemia (including atherogenic dyslipidaemia), related metabolic disorders and cardiovascular disease.

Preferably the CBD and THCV are in a predefined ratio by weight.

Preferably the pharmaceutical or neutraceutical formulation is used in combination with one or more other drugs used in the treatment of diabetes, obesity, dyslipidaemia (including atherogenic dyslipidaemia), related metabolic disorders or cardiovascular disease.

More preferably the one or other drugs is either a drug to reduce the insulin resistance or enhance secretion or a combination of the two.

According to a second aspect of the present invention there is provided the use of THCV alone or in combination with another cannabinoid, in the manufacture of a pharmaceutical or neutraceutical formulation for use in increasing energy expenditure in a subject.

Preferably the pharmaceutical or neutraceutical formulation is packaged for use for an extended period.

More preferably the extended period is at least 10 days.

Preferably the other cannabinoid is CBD.

Preferably the THCV is in the form of a cannabinoid-containing plant extract derived from at least one cannabis plant.

More preferably the cannabinoid-containing plant extract derived from at least one cannabis plant is a botanical drug substance.

More preferably the cannabinoid-containing plant extract from at least one cannabis plant comprises all or some of the naturally occurring cannabinoids in the plant.

More preferably still, all or a significant amount of any THC occurring in the cannabis-containing plant extract has been removed.

Alternatively the THCV or any other cannabinoid is/are in a substantially pure or isolated form.

A “substantially pure” preparation of cannabinoid is defined as a preparation having a chromatographic purity (of the desired cannabinoid) of greater than 90%, more preferably greater than 95%, more preferably greater than 96%, more preferably greater than 97%, more preferably greater than 98%, more preferably greater than 99% and most preferably greater than 99.5%, as determined by area normalisation of an HPLC profile.

Preferably the substantially pure cannabinoid used in the invention is substantially free of any other naturally occurring or synthetic cannabinoids, including cannabinoids which occur naturally in cannabis plants. In this context “substantially free” can be taken to mean that no cannabinoids other than the target cannabinoid are detectable by HPLC.

Particularly in the case of THCV, it is known that the cannabinoid THCV is produced together with THC in the cannabis plant. The psychoactive side-effects of THC are not wanted especially when the THCV is to be used in a pharmaceutical formulation and as such the plant extracts used in the formulations of the invention can be selectively treated to remove other cannabinoids such as THC.

Alternatively the THCV or any other cannabinoid is/are in a synthetic form.

Preferably the THCV is present in a dose effective to bring about an increase in energy expenditure in a subject.

Preferably the effective dose of THCV is between 0.3 mg/kg and 30.0 mg/kg.

Preferably when CBD is present, the dose of CBD is between 0.1 mg/kg and 5.0 mg/kg.

Preferably the increase in energy expenditure forms part of a regime to manage or treat type I or II diabetes, obesity, dyslipidaemiac (including atherogenic dyslipidaemia), related metabolic disorders and cardiovascular disease.

More preferably the pharmaceutical or neutraceutical formulation modulates the levels of one or more of leptin and/or adiponectin.

Preferably the CBD and THCV are in a predefined ratio by weight.

Preferably the pharmaceutical or neutraceutical formulation is used in combination with one or more other drugs used in the treatment of diabetes, obesity, dyslipidaemia (including atherogenic dyslipidaemia), related metabolic disorders or cardiovascular disease.

More preferably the THCV is used to treat obesity and the other drug is used to either reduce food intake or alter metabolism.

A third aspect of the present invention there is provided a method of controlling cholesterol levels in a subject, comprising administering to a subject in need thereof an effective amount of CBD alone or in combination with another cannabinoid.

A fourth aspect of the present invention there is provided a method of increasing energy expenditure in a subject, comprising administering to a subject in need thereof an effective amount of THCV alone or in combination with another cannabinoid.

Disclosed herein are data describing the use of the cannabinoids THCV and/or CBD, alone and in combination in various models of diabetes. These data show a reduction in the percentage of body fat, an increase in energy expenditure, a reduction in the total cholesterol levels and an increase in HDL (“good”) cholesterol levels. In addition these data from show desirable effects on plasma insulin, leptin and adiponectin levels.

Additionally it is disclosed herein for the first time that CBD acts as a PPAR gamma ligand. Accordingly this mechanism of action supports the data that CBD alone is of use in the prevention or treatment of diabetes, obesity and related metabolic disorders.

Certain aspects of this invention are further described, by way of example only, with reference to the accompanying drawings in which:

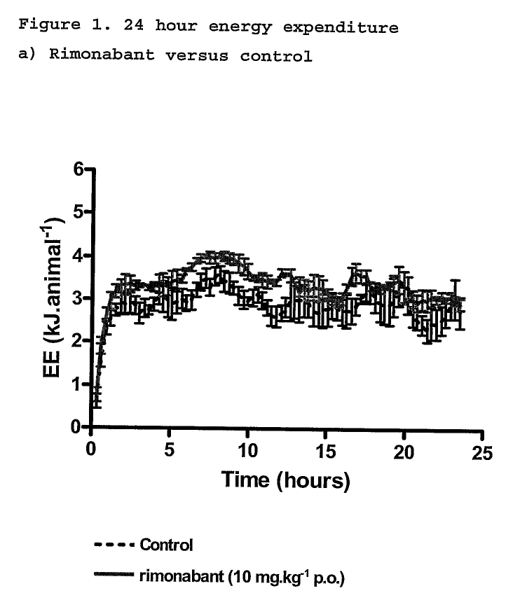

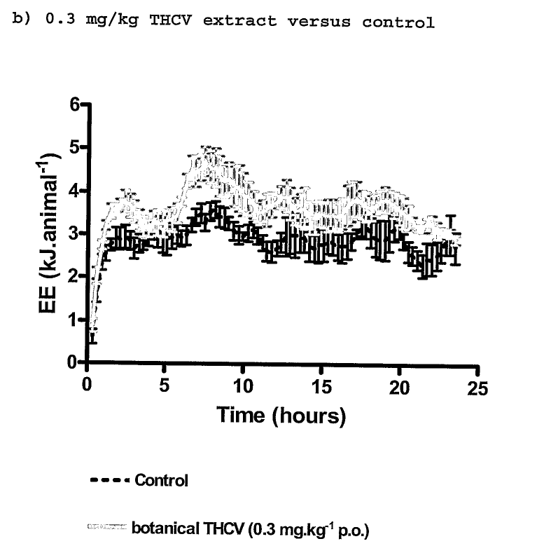

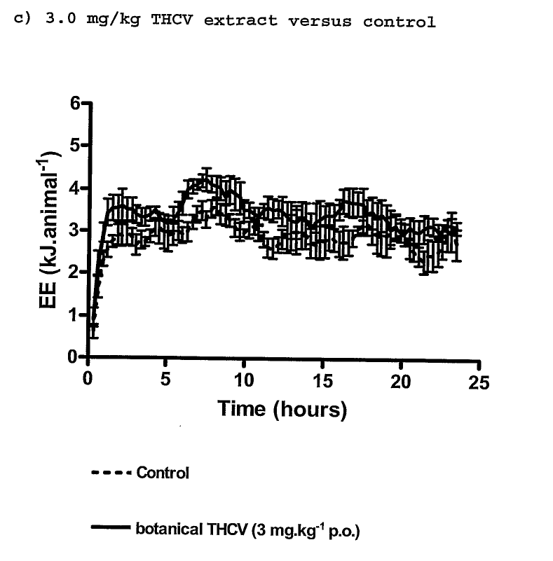

FIG. 1 (a to e) shows the 24 hour energy expenditure of test articles versus the control;

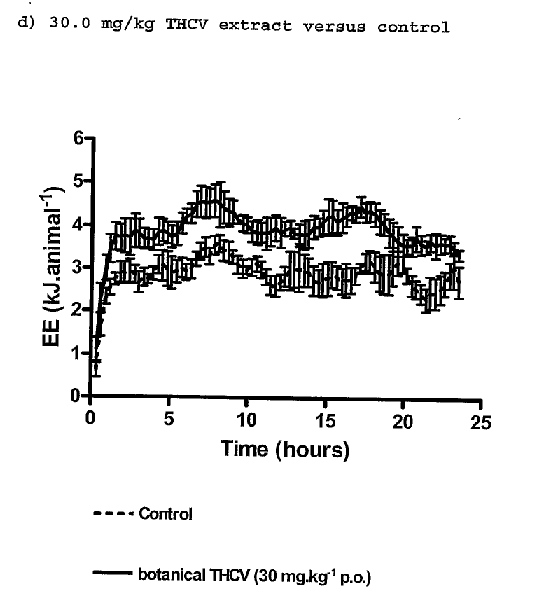

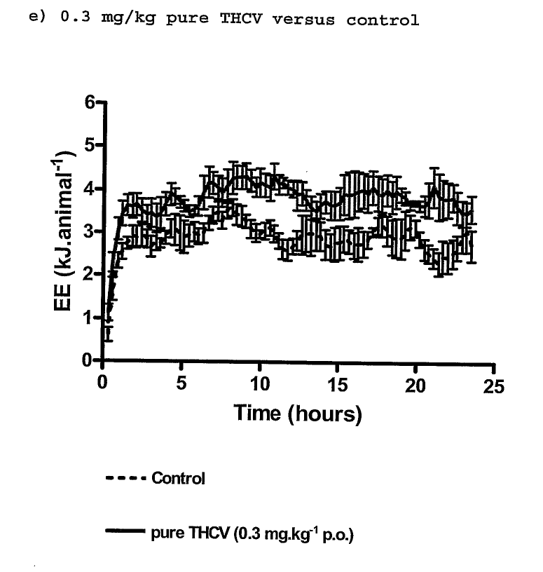

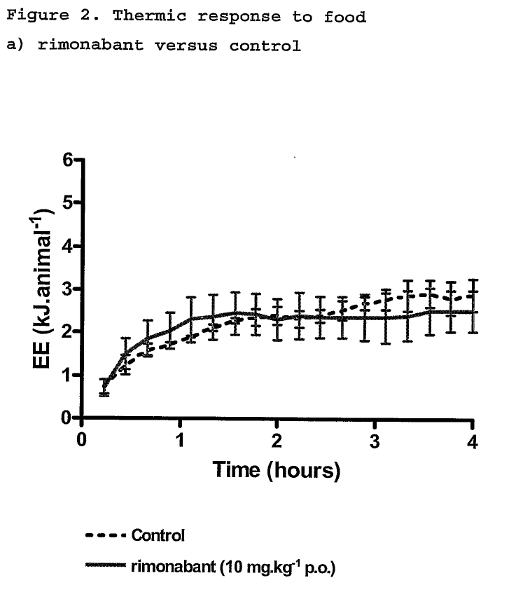

FIG. 2 (a to e) shows the thermic response to food - 3 hour energy expenditure;

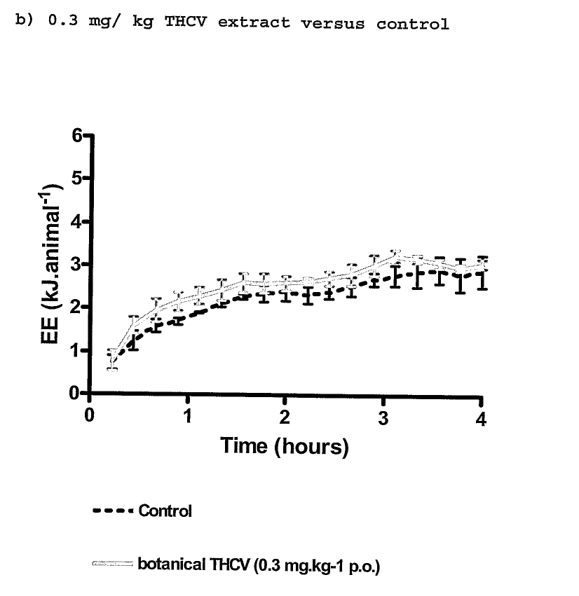

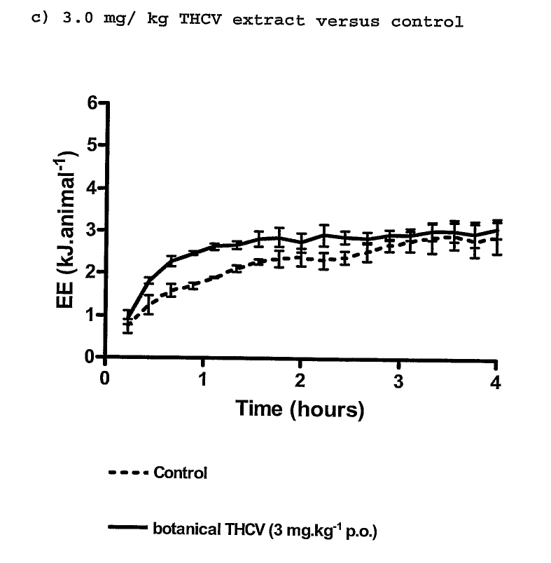

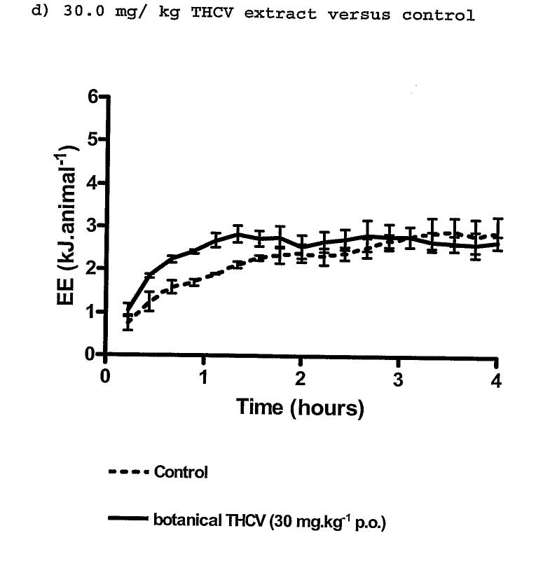

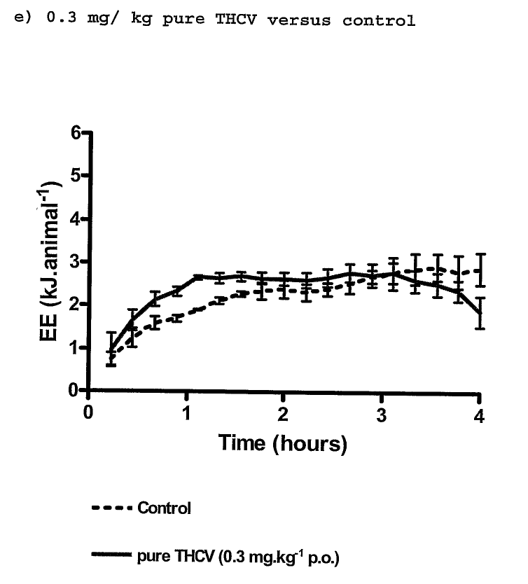

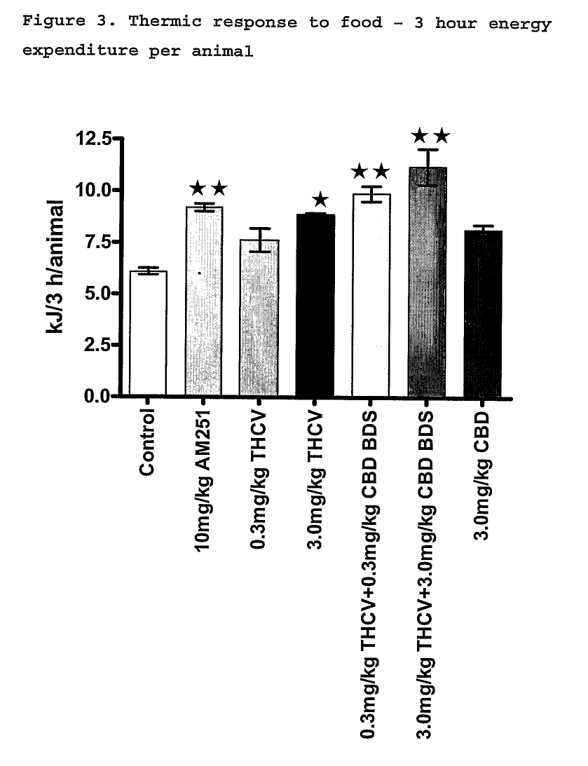

FIG. 3 shows the thermic response to food;

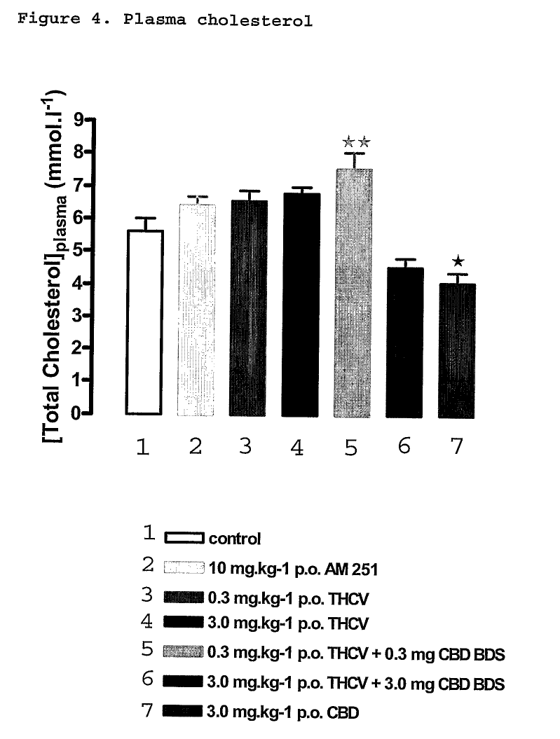

FIG. 4 shows the plasma cholesterol levels;

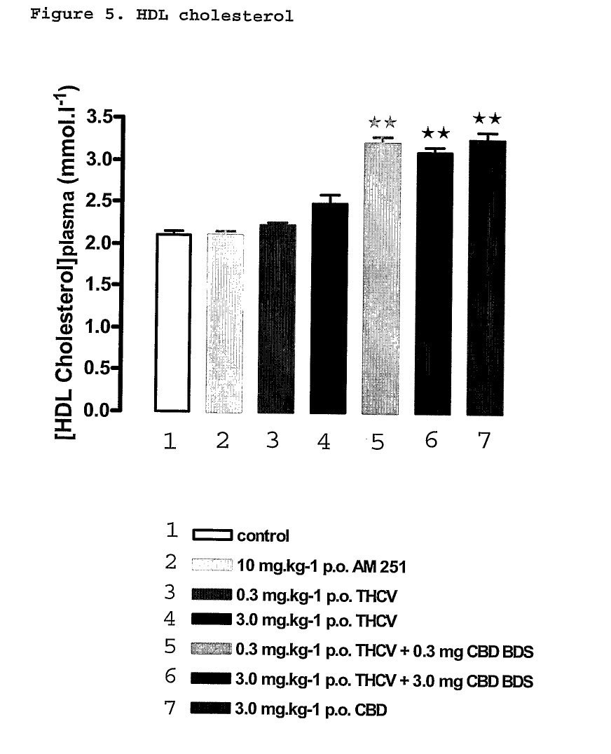

FIG. 5 shows the HDL cholesterol levels;

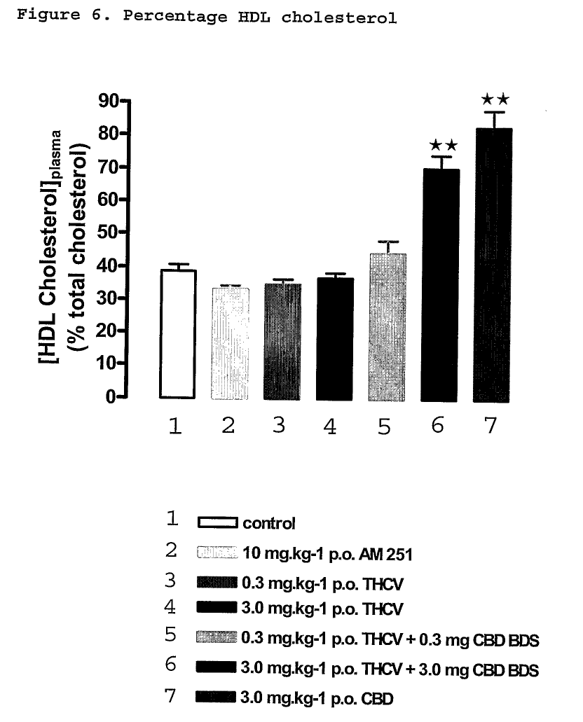

FIG. 6 shows the percentage HDL cholesterol levels; and

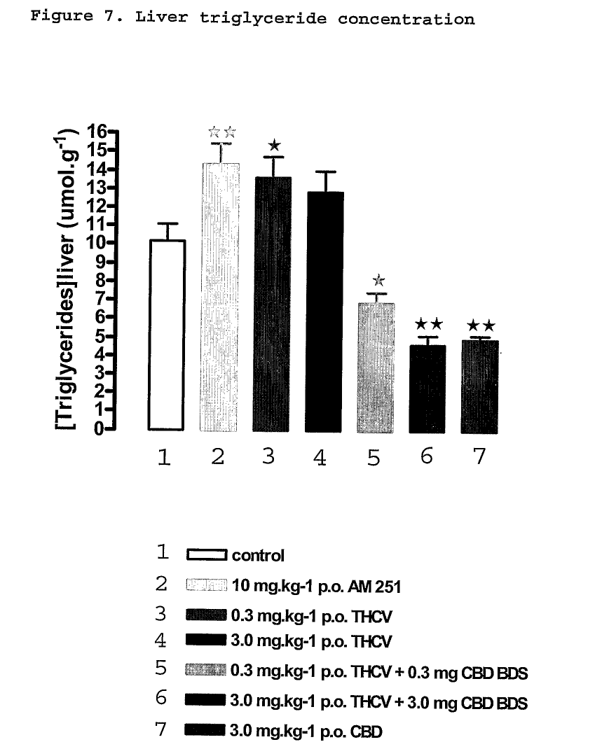

FIG. 7 shows the liver triglyceride concentration.

### SPECIFIC DESCRIPTION

Examples 1 and 2 below describe the use of the cannabinoids THCV and CBD in a series of test models. Example 3 describes data derived from similar experiments using a combination of the cannabinoids THCV and CBD and a different animal model. Example 4 discloses the mechanism of action of CBD acting as a PPAR gamma ligand.

The following are a battery of tests used to elicit the effects of drugs on diseases including type I or II diabetes, obesity, dyslipidaemia, related metabolic disorders and cardiovascular disease.
a) Body weight: Bodyweights of all mice were measured twice weekly during the study.
b) Food intake: Food intake was measured by weight difference taking account of food wastage in the bottom of the cage.
c) Measurement of water consumption: Water intake was calculated daily for each animal, during the treatment period, by weight difference of the water bottle.
d) Oral Glucose Tolerance Test (OGTT): Glucose was dissolved in water (3 g/10 ml) and given to the mice orally at a rate of 3 g/kg. Blood samples (20 μl) were taken for the analysis of glucose concentration at −30, 0, 30, 60, 90, 120 and 180 minutes following glucose administration. Blood samples were also taken at −30 minutes for insulin analysis.
e) Blood Glucose analysis: Duplicate 20 μl samples of blood of were taken for each individual sample and placed in a 96-well assay plate. To each well was added 180 μl aliquots of glucose oxidase reagent. Samples were mixed and then left for approximately 30 minutes. Samples were then analysed automatically using a SpectraMax-250 and SoftMax Pro software.
f) Plasma preparation: Blood was collected for the measurement of i) plasma insulin, ii) cholesterol, iii) free fatty acids or iv) triglyceride concentration.
   i. Plasma insulin analysis: Plasma insulin was measured using 5 μl of plasma, compared with a mouse insulin standard using a 96-well micro-assay plate
   ii. Plasma cholesterol analysis: To each sample was added 200 μl of infinity cholesterol liquid stable reagent. The samples were mixed and incubated for 5 minutes before reading at dual wavelengths of 500 and 660 nm.
   iii. Plasma free fatty acids: 5 μl samples of plasma were measured into a 96-well assay plate, to which was added 0.2 ml of reagents NEFA C
   iv. Plasma triglycerides analysis: To each well 200 μl aliquots of triglycerides reagent. The samples were mixed and then left for approximately 45 minutes before measurement.
g) Measurement of body fat: Whole body fat was measured utilising Dual Energy X-ray Absorptiometry (DEXA), which measures the difference in absorption of body tissue at two X-ray energies. Comparison with a calibrated standard allowed identification of types of tissue by their density. Dedicated software was using to quantify amounts of bone and fat. The mice were lightly anaesthetised, sufficient to keep them quiescent during this non-invasive technique.
h) Energy expenditure measurements: Energy expenditure was measured by open circuit indirect calorimetry with mice in their home cages. For studies of 24 h energy expenditure, mice were dosed with their allocated treatment and then measurements commenced. For measurements of the thermic effect of food, the mice were dosed orally with complan (Complan Foods Ltd., by energy 18% protein, 53% carbohydrate, 29% fat) 10 g kg⁻¹=185 kJ kg⁻¹.
i) Plasma leptin: leptin plays a key role in regulating energy intake and energy expenditure, including the decrease of appetite and increase of metabolism. Plasma leptin was measured in fed mice using the Crystal Chem Elisa assay.
j) Plasma adiponectin: Adiponectin exerts some of its weight reduction effects via the brain. This is similar to the action of leptin, but the two hormones perform complementary actions, and can have additive effects. Plasma adiponectin was measured in overnight fasted mice using the 96-well B-Bridge Elisa assay.
k) Plasma HDL-cholesterol: Plasma HDL-cholesterol was measured in fed mice using the kit from Trinity Biotech.
l) 24 h blood glucose profile: This was determined in order to ensure that changes in the diurnal control of blood glucose were detected. Thus blood samples were obtained at three hourly intervals throughout a continuous 24 h period.
m) Plasma haemoglobin A_{IC}: Samples (10 μl) were analysed using the A_{IC}. This uses a micro-optical detection method that incorporates microelectronics, optics and dry reagent chemistry within a self contained single use monitor: HbA_{IC} is conjugated to anti HbA_{IC} antibodies bound to blue microparticles and concentration determined by reflectance of the blue colour at 618 nM. For total haemoglobin, ferricyanide converts haemoglobin to methaemoglobin and the concentration measured from the reflectance of the orange-brown colour at 565 nM.
n) Pancreatic insulin: Frozen pancreas were weighed and mashed in a small size pecel in liquid nitrogen. The homogenate was then extracted overnight at 4° C. and centrifuged at 2000 g for 5 min.
o) Pancreatic islet histology: 4 μM sections were cut using a Leica RM2125 rotary microtome, transferred to positively-charged glass slides and dried overnight at 42° C. Morphology was assessed using hematoxylin and eosin staining.
p) Liver glycogen: After centrifugation (3000×g for 15 min), an aliquot of the supernatant (10 μl) is assayed using the glucose oxidase method. Results are expressed as glucosyl units/g liver.
q) Liver triglyceride: 15-30 mg samples of liver were homogenised in 500 μl methanol using a Ribolyser cell disruptor at 4° C. 1 ml of chloroform is added and tubes vortexed and left at 4° C. for 2 h with vortexing every 30 min. 200 μl of 0.9% sodium chloride is added and after thorough vortexing, the mixture is centrifuged at 300×g for 5 min. A 500 μl aliquot of the chloroform phase is taken and chloroform removed by evaporation. The residue is dissolved in 200 μl ethanol and triglyceride content measured.

The statistical significance of any differences between control animals and treated animals was determined using ANOVA tests. Statistical significance compared with data from the group given vehicle alone is shown as: * p<0.05, ** p<0.01 or *** p<0.001.

Key results from these tests are highlighted in Examples 1 to 3 below.

### EXAMPLE 1

### The Effect of Tetrahydrocannabivarin in Diabetes, Obesity and Related Metabolic Disorders

CB₁ receptor antagonists are being examined as potential anti-obesity agents and the compound, rimonabant, is marketed in a number of European countries.

Rimonabant shows anti-obesity effects in man and rodent models. In rodent models, rimonabant reduces food intake over the first few days but the long term anti-obesity effects seem to be largely independent of food intake reduction. It seems likely that the anti-obesity effect in the long term relates more to increases in energy expenditure, possibly mediated via increased release of adiponectin from adipose tissue.

Tetrahydrocannabivarin (THCV), an analogue of Δ9-tetrahydrocannabinol with a 3-propyl instead of a 3-pentyl side chain, is a natural product with significant activity at the CB₁ receptor. The example described herein was designed to examine the potential of THCV both as a botanical (also referred to as cannabis-based plant extract), which also contains THC, and as pure substance in the dietary induced obese (DIO) mouse model.

The C57B1/6 mouse fed on a high fat diet for around 12 weeks is a standard model used to evaluate agents likely to affect metabolic disease including obesity, type II diabetes and dyslipidaemia. Thus both potential anti-obesity effects and effects on diabetic and dyslipidaemia parameters will be measured.

### Methods:

Animals were given SDS diet 829100, which contains 0.2% cholesterol ad hoc and a 12 hour light cycle (lights on 07:00).

Seventy mice were selected that showed good weight gain on the diet and were placed into cages of 4 mice each.

After a few days rest to adapt to new environment and caging, an acute food intake study was undertaken as follows:

A control
B 1 mg·kg⁻¹ p.o. THCV (Botanical)
C 3 mg·kg⁻¹ p.o. THCV (Botanical)
D 10 mg·kg⁻¹ p.o. THCV (Botanical)
E 30 mg·kg⁻¹ p.o. THCV (Botanical)

The mice were grouped in three groups of four mice per study group.

The compounds to be dosed just before lights out (19:00) and food intake measured at 2 h, 4 h and 24 h.

A chronic dosing study was then undertaken as follows:

A control
B rimonabant (10 mg·kg⁻¹ p.a.)
C AM 251 (10 mg·kg⁻¹ p.o.)
D botanical THCV (0.3 mg·kg⁻¹ p.o.)
E botanical THCV (3 mg·kg⁻¹ p.o.)
F botanical THCV (30 mg·kg⁻¹ p.o.)
G pure THCV (0.3 mg·kg⁻¹ p.o.)

Once the acute studies were complete the mice were grouped in two groups of five mice for the chronic dosing study. Dosing was daily by oral gavage at 09:00-10:00.

Measurements were taken throughout the study as follows:
Daily: Food and water intake
Twice weekly: Body weight
Days 3, 4 and 5: 24 hour energy expenditure starting immediately after dosing
Days 7 or 8: Oral glucose tolerance (glucose load 3 g·kg⁻¹) in 5 h-fased mice measuring glucose at −30, 0, 30, 60, 90, 120 and insulin at −30
Days 10, 11 and 12: 24 hour energy expenditure starting immediately after dosing.
Days 15, 16 and 17: Thermic response to a mixed meal, fast mice for 2 h, dose mice then 60 mins later give a 10 g·kg⁻¹ Complan® meal (orally), measure energy expenditure from dosing for 4 h post complan meal
Day 21: Oral glucose tolerance in 5 h-fasted mice (repeat day 7 study
Day 28: body composition by Dexascan in anaesthetised mice, measure nose-anus length, blood sample from fed mice for glucose, lactate, insulin, triglycerides, total cholesterol, HDL-cholesterol, leptin
Day 29: Fast overnight
Day 30: Blood sample for glucose, free fatty acids, insulin and adiponectin dose mice and take blood sample 2-3 h post-dosing for drug levels
Day 30: Termination

### Key Results:

### Effect on 24 h Energy Expenditure (Days 10-12)

Rimonabant showed a small increase in energy expenditure relative to control in the first 12 h post-dosing. THCV-BDS at the high dose of 30 mg kg⁻¹ increased energy expenditure throughout the 24 h period but the lower doses of 0.3 and 3.0 mg kg⁻¹ were less effective. Pure THCV (0.3 mg kg⁻¹) had a similar level of effect to rimonabant. Table 1 describes these results along with FIGS. 1 a) to e).

TABLE 1
Energy expenditure at days 10-12

| | | | | | | | | | | botanical THCV | | |
|---|---|---|---|---|---|---|---|---|---|---|---|---|
| | | | | rimonabant | | | AM 251 | | | (0.3 mg · kg−1 | | |
| Day | Control | | | (10 mg · kg−1 p.o.) | | | (10 mg · kg−1 p.o.) | | | p.o.) | | |
| 0.30 | 0.29 | 0.85 | 0.73 | 0.43 | 1.01 | 0.86 | 0.46 | 1.15 | 0.91 | 0.58 | 1.06 | 0.96 |
| 0.60 | 1.13 | 1.96 | 1.96 | 1.50 | 2.16 | 2.05 | 1.68 | 2.61 | 2.56 | 1.66 | 2.30 | 2.10 |
| 0.90 | 1.98 | 2.53 | 2.49 | 2.34 | 2.69 | 2.86 | 2.77 | 3.22 | 3.55 | 2.57 | 2.86 | 2.82 |
| 1.21 | 2.21 | 2.81 | 2.66 | 2.55 | 3.15 | 3.18 | 3.29 | 3.88 | 3.91 | 2.98 | 3.33 | 3.30 |
| 1.51 | 2.50 | 2.91 | 2.85 | 2.90 | 3.26 | 3.38 | 3.67 | 4.02 | 4.18 | 3.26 | 3.53 | 3.80 |
| 1.81 | 2.47 | 3.29 | 3.01 | 3.06 | 3.48 | 3.45 | 3.81 | 4.34 | 4.27 | 3.50 | 3.73 | 3.67 |
| 2.11 | 2.42 | 3.23 | 3.03 | 2.87 | 3.53 | 3.60 | 3.85 | 4.27 | 4.44 | 3.91 | 3.53 | 3.67 |
| 2.41 | 2.41 | 3.24 | 3.19 | 2.98 | 3.43 | 3.65 | 3.94 | 4.06 | 4.44 | 4.25 | 3.38 | 3.67 |
| 2.71 | 2.46 | 3.06 | 3.05 | 3.09 | 3.41 | 3.44 | 4.06 | 4.01 | 4.48 | 4.13 | 3.19 | 3.70 |
| 3.01 | 2.25 | 3.07 | 2.72 | 3.24 | 3.33 | 3.41 | 3.96 | 4.47 | 4.45 | 3.86 | 3.06 | 3.68 |
| 3.31 | 2.42 | 3.16 | 2.79 | 3.34 | 3.35 | 3.34 | 3.98 | 4.15 | 4.56 | 3.70 | 2.86 | 3.44 |
| 3.62 | 2.66 | 2.96 | 2.66 | 3.33 | 3.31 | 3.18 | 3.91 | 4.32 | 4.14 | 3.63 | 2.78 | 3.14 |
| 3.92 | 2.57 | 2.99 | 3.10 | 3.08 | 3.33 | 3.48 | 4.35 | 4.36 | 4.08 | 3.49 | 2.80 | 3.10 |
| 4.22 | 2.67 | 3.34 | 3.25 | 3.02 | 3.37 | 3.70 | 4.05 | 4.42 | 4.28 | 3.48 | 2.84 | 3.60 |
| 4.52 | 2.36 | 3.39 | 3.47 | 2.85 | 3.41 | 3.75 | 3.74 | 4.25 | 4.40 | 3.72 | 2.72 | 3.38 |
| 4.82 | 2.21 | 3.58 | 3.19 | 2.88 | 3.45 | 3.59 | 3.65 | 4.30 | 3.98 | 3.82 | 2.76 | 3.19 |
| 5.12 | 2.24 | 3.57 | 2.88 | 2.97 | 3.76 | 3.58 | 3.69 | 4.52 | 4.22 | 3.77 | 3.05 | 3.17 |
| 5.42 | 2.52 | 3.39 | 2.91 | 3.03 | 3.75 | 3.61 | 3.60 | 4.73 | 4.27 | 3.93 | 3.05 | 3.07 |
| 5.72 | 2.65 | 3.40 | 3.02 | 3.32 | 3.63 | 3.50 | 3.83 | 4.96 | 4.35 | 3.94 | 3.38 | 3.18 |
| 6.03 | 2.64 | 3.28 | 2.83 | 3.62 | 3.74 | 3.69 | 4.03 | 4.79 | 4.38 | 3.94 | 3.57 | 3.79 |
| 6.33 | 2.74 | 3.43 | 2.81 | 3.76 | 3.90 | 3.61 | 4.12 | 5.25 | 4.59 | 4.56 | 4.05 | 4.09 |
| 6.63 | 3.00 | 3.58 | 3.12 | 4.03 | 3.86 | 3.82 | 4.56 | 5.68 | 4.78 | 5.13 | 4.19 | 4.32 |
| 6.93 | 3.19 | 3.80 | 3.25 | 4.04 | 4.09 | 3.79 | 5.04 | 5.65 | 4.82 | 5.03 | 3.93 | 4.37 |
| 7.23 | 3.20 | 3.91 | 3.37 | 4.05 | 4.06 | 3.92 | 5.21 | 5.39 | 4.70 | 5.11 | 4.15 | 4.43 |
| 7.53 | 3.10 | 3.69 | 3.07 | 3.95 | 4.21 | 3.78 | 5.22 | 5.41 | 4.78 | 5.33 | 4.14 | 4.68 |
| 7.83 | 3.29 | 3.96 | 3.38 | 3.97 | 4.19 | 3.77 | 5.06 | 5.22 | 4.94 | 5.15 | 3.96 | 4.50 |
| 8.13 | 3.31 | 3.95 | 3.51 | 4.00 | 4.15 | 3.95 | 5.16 | 5.06 | 4.98 | 5.41 | 3.92 | 4.37 |
| 8.44 | 3.29 | 3.90 | 3.10 | 4.13 | 4.10 | 3.80 | 5.06 | 4.75 | 4.99 | 5.30 | 3.87 | 3.92 |
| 8.74 | 3.08 | 3.86 | 3.27 | 4.18 | 3.90 | 3.81 | 4.90 | 4.69 | 5.23 | 4.97 | 3.83 | 3.91 |
| 9.04 | 3.01 | 3.52 | 3.30 | 4.18 | 3.88 | 3.75 | 5.02 | 4.79 | 4.82 | 5.06 | 3.81 | 4.10 |
| 9.34 | 2.83 | 3.52 | 2.94 | 4.19 | 3.88 | 3.47 | 5.18 | 4.85 | 4.77 | 5.04 | 3.49 | 3.90 |
| 9.64 | 2.73 | 3.41 | 2.87 | 4.06 | 3.86 | 3.40 | 5.14 | 4.83 | 4.56 | 5.07 | 3.22 | 3.99 |
| 9.94 | 2.91 | 3.17 | 2.85 | 3.98 | 3.64 | 3.38 | 5.14 | 5.26 | 4.60 | 4.92 | 3.26 | 3.67 |
| 10.24 | 2.83 | 3.44 | 2.89 | 3.50 | 3.63 | 3.43 | 4.93 | 5.56 | 4.70 | 4.53 | 3.25 | 3.85 |
| 10.54 | 2.84 | 3.45 | 3.08 | 3.48 | 3.64 | 3.36 | 4.66 | 5.03 | 4.50 | 4.14 | 3.10 | 3.88 |
| 10.85 | 2.64 | 3.06 | 3.26 | 3.31 | 3.77 | 3.33 | 4.42 | 4.95 | 4.31 | 3.94 | 3.19 | 3.42 |
| 11.15 | 2.48 | 2.87 | 3.10 | 3.23 | 3.71 | 3.31 | 4.42 | 4.66 | 4.09 | 3.86 | 3.04 | 3.77 |
| 11.45 | 2.24 | 2.69 | 2.96 | 3.40 | 3.53 | 3.38 | 4.74 | 4.50 | 4.11 | 3.86 | 2.83 | 4.09 |
| 11.75 | 2.22 | 3.02 | 2.61 | 3.44 | 3.54 | 3.28 | 4.76 | 4.64 | 4.02 | 3.88 | 3.16 | 4.09 |
| 12.05 | 2.51 | 3.05 | 2.55 | 3.57 | 3.83 | 3.49 | 4.75 | 4.69 | 4.20 | 3.87 | 3.49 | 3.99 |
| 12.35 | 2.56 | 3.27 | 2.65 | 3.56 | 3.65 | 3.58 | 5.12 | 4.53 | 4.25 | 4.25 | 3.39 | 4.00 |
| 12.65 | 2.24 | 3.81 | 2.87 | 3.49 | 3.85 | 3.36 | 5.07 | 4.50 | 4.08 | 4.52 | 3.22 | 4.01 |
| 12.96 | 2.20 | 3.57 | 3.25 | 3.39 | 3.60 | 3.28 | 4.95 | 4.27 | 4.11 | 4.31 | 3.06 | 3.87 |
| 13.26 | 1.97 | 3.69 | 3.39 | 3.08 | 3.40 | 3.11 | 4.48 | 4.01 | 4.08 | 3.91 | 2.94 | 3.85 |
| 13.56 | 2.24 | 3.50 | 3.21 | 2.96 | 3.64 | 3.28 | 4.40 | 3.97 | 4.09 | 3.91 | 2.83 | 4.03 |
| 13.86 | 2.27 | 3.33 | 3.22 | 2.77 | 3.52 | 3.38 | 4.53 | 3.93 | 4.02 | 4.05 | 2.67 | 4.18 |
| 14.16 | 2.07 | 3.16 | 3.04 | 2.74 | 3.74 | 3.41 | 4.42 | 3.75 | 3.99 | 3.85 | 2.65 | 3.73 |
| 14.46 | 2.13 | 3.16 | 2.85 | 2.80 | 3.83 | 3.22 | 4.48 | 3.91 | 4.15 | 3.91 | 2.67 | 3.65 |
| 14.76 | 2.01 | 3.20 | 3.09 | 2.85 | 3.56 | 3.38 | 4.38 | 4.21 | 4.39 | 3.97 | 2.96 | 3.64 |
| 15.06 | 2.06 | 3.39 | 2.95 | 2.89 | 3.38 | 3.16 | 4.41 | 4.19 | 4.09 | 4.00 | 3.00 | 3.43 |
| 15.37 | 2.33 | 3.33 | 2.98 | 2.85 | 3.20 | 3.04 | 4.37 | 4.38 | 4.38 | 4.12 | 2.85 | 3.44 |
| 15.67 | 2.43 | 3.35 | 2.71 | 2.79 | 3.05 | 2.99 | 4.15 | 4.19 | 4.35 | 4.06 | 2.85 | 3.53 |
| 15.97 | 2.20 | 3.28 | 2.72 | 2.92 | 3.33 | 2.92 | 3.97 | 4.34 | 5.14 | 4.03 | 2.87 | 3.38 |
| 16.27 | 2.11 | 3.45 | 2.74 | 3.27 | 3.49 | 3.11 | 3.99 | 4.10 | 5.17 | 4.21 | 2.95 | 3.38 |
| 16.57 | 2.28 | 3.32 | 2.71 | 3.77 | 3.80 | 3.14 | 4.32 | 4.08 | 4.57 | 4.53 | 2.93 | 3.67 |
| 16.87 | 2.68 | 3.24 | 2.70 | 3.98 | 3.76 | 3.23 | 4.58 | 3.82 | 4.59 | 4.65 | 3.05 | 3.67 |
| 17.17 | 2.88 | 3.27 | 3.00 | 3.56 | 3.84 | 3.33 | 4.74 | 3.92 | 4.39 | 4.66 | 3.29 | 3.64 |
| 17.47 | 2.86 | 3.18 | 3.54 | 3.87 | 3.54 | 3.35 | 4.88 | 3.95 | 4.35 | 4.35 | 3.20 | 3.62 |
| 17.78 | 2.70 | 3.35 | 3.56 | 3.54 | 3.38 | 3.16 | 4.89 | 4.42 | 4.28 | 4.32 | 3.16 | 3.57 |
| 18.08 | 2.42 | 3.50 | 3.32 | 3.30 | 3.20 | 3.21 | 4.86 | 4.63 | 4.62 | 4.13 | 3.15 | 3.64 |
| 18.38 | 2.12 | 3.33 | 3.35 | 3.44 | 3.32 | 3.08 | 4.79 | 4.51 | 4.67 | 4.06 | 3.30 | 3.83 |
| 18.68 | 2.00 | 3.32 | 3.39 | 3.39 | 3.31 | 3.42 | 4.66 | 4.52 | 4.72 | 3.77 | 3.42 | 4.27 |
| 18.98 | 1.99 | 3.53 | 3.20 | 3.30 | 3.52 | 3.25 | 4.49 | 4.62 | 4.83 | 3.92 | 3.30 | 4.32 |
| 19.28 | 2.20 | 3.49 | 3.57 | 3.52 | 3.69 | 3.05 | 4.55 | 4.38 | 4.63 | 4.14 | 3.30 | 3.97 |
| 19.58 | 2.67 | 3.65 | 3.17 | 3.75 | 3.85 | 2.88 | 4.61 | 4.16 | 4.63 | 4.21 | 3.16 | 3.81 |
| 19.88 | 2.92 | 3.53 | 2.97 | 3.43 | 3.89 | 2.68 | 4.62 | 4.21 | 4.57 | 4.21 | 3.03 | 3.61 |
| 20.19 | 2.77 | 3.22 | 2.62 | 3.27 | 3.79 | 2.82 | 4.57 | 4.20 | 4.53 | 4.07 | 2.85 | 3.74 |
| 20.49 | 2.20 | 3.04 | 2.71 | 2.78 | 3.44 | 2.91 | 4.33 | 4.66 | 4.67 | 3.64 | 2.85 | 3.82 |
| 20.79 | 2.15 | 2.84 | 2.70 | 2.78 | 3.36 | 2.74 | 4.02 | 4.69 | 4.58 | 3.44 | 3.14 | 3.47 |
| 21.09 | 2.02 | 2.64 | 2.71 | 2.69 | 3.48 | 2.92 | 4.05 | 5.03 | 4.45 | 3.18 | 3.10 | 3.34 |
| 21.39 | 1.83 | 2.77 | 2.38 | 2.65 | 3.45 | 2.90 | 3.74 | 4.96 | 4.42 | 3.12 | 3.07 | 3.35 |
| 21.69 | 1.88 | 3.15 | 2.39 | 2.86 | 3.49 | 2.92 | 3.84 | 4.82 | 4.21 | 3.49 | 3.28 | 3.20 |
| 21.99 | 1.90 | 3.13 | 2.37 | 3.05 | 3.17 | 2.89 | 3.73 | 4.45 | 4.40 | 3.68 | 3.10 | 3.06 |
| 22.29 | 2.06 | 3.16 | 2.44 | 3.22 | 3.19 | 2.96 | 3.75 | 4.38 | 4.27 | 3.52 | 2.98 | 2.92 |
| 22.60 | 2.16 | 3.15 | 2.75 | 3.15 | 3.02 | 2.95 | 3.88 | 4.36 | 4.25 | 3.31 | 2.96 | 3.07 |
| 22.90 | 2.30 | 3.15 | 3.23 | 2.96 | 3.34 | 2.98 | 3.53 | 4.59 | 4.25 | 3.10 | 2.96 | 3.23 |
| 23.20 | 2.18 | 3.70 | 3.28 | 2.84 | 3.17 | 3.06 | 3.42 | 4.82 | 4.07 | 3.29 | 2.96 | 2.97 |
| 23.50 | 1.99 | 3.08 | 3.12 | 2.65 | 3.10 | 3.00 | 3.34 | 4.62 | 3.64 | 3.12 | 2.89 | 2.80 |

| | botanical THCV | | | botanical THCV | | | pure THCV | | |
|---|---|---|---|---|---|---|---|---|---|
| Day | (3 mg · kg−1 p.o.) | | | (3 mg · kg−1 p.o.) | | | (0.3 mg · kg−1 p.o.) | | |
| 0.30 | 0.61 | 1.34 | 0.89 | 0.61 | 1.34 | 0.89 | 0.61 | 1.34 | 0.89 |
| 0.60 | 1.65 | 2.60 | 2.44 | 1.65 | 2.60 | 2.44 | 1.65 | 2.60 | 2.44 |
| 0.90 | 2.50 | 3.12 | 3.24 | 2.50 | 3.12 | 3.24 | 2.50 | 3.12 | 3.24 |
| 1.21 | 2.83 | 3.56 | 3.91 | 2.83 | 3.56 | 3.91 | 2.83 | 3.56 | 3.91 |
| 1.51 | 2.98 | 3.66 | 3.98 | 2.98 | 3.66 | 3.98 | 2.98 | 3.66 | 3.98 |
| 1.81 | 2.94 | 3.58 | 4.07 | 2.94 | 3.58 | 4.07 | 2.94 | 3.58 | 4.07 |
| 2.11 | 2.93 | 3.66 | 4.25 | 2.93 | 3.66 | 4.25 | 2.93 | 3.66 | 4.25 |
| 2.41 | 2.99 | 3.52 | 4.01 | 2.99 | 3.52 | 4.01 | 2.99 | 3.52 | 4.01 |
| 2.71 | 3.15 | 3.33 | 4.06 | 3.15 | 3.33 | 4.06 | 3.15 | 3.33 | 4.06 |
| 3.01 | 3.20 | 3.20 | 3.79 | 3.20 | 3.20 | 3.79 | 3.20 | 3.20 | 3.79 |
| 3.31 | 3.38 | 2.95 | 3.69 | 3.38 | 2.95 | 3.69 | 3.38 | 2.95 | 3.69 |
| 3.62 | 3.48 | 2.92 | 3.63 | 3.48 | 2.92 | 3.63 | 3.48 | 2.92 | 3.63 |
| 3.92 | 3.48 | 3.10 | 3.65 | 3.48 | 3.10 | 3.65 | 3.48 | 3.10 | 3.65 |
| 4.22 | 3.46 | 3.45 | 3.62 | 3.46 | 3.45 | 3.62 | 3.46 | 3.45 | 3.62 |
| 4.52 | 3.19 | 3.34 | 3.51 | 3.19 | 3.34 | 3.51 | 3.19 | 3.34 | 3.51 |
| 4.82 | 3.16 | 2.90 | 3.54 | 3.16 | 2.90 | 3.54 | 3.16 | 2.90 | 3.54 |
| 5.12 | 3.10 | 2.89 | 3.56 | 3.10 | 2.89 | 3.56 | 3.10 | 2.89 | 3.56 |
| 5.42 | 2.99 | 3.02 | 3.84 | 2.99 | 3.02 | 3.84 | 2.99 | 3.02 | 3.84 |
| 5.72 | 3.28 | 3.19 | 3.92 | 3.28 | 3.19 | 3.92 | 3.28 | 3.19 | 3.92 |
| 6.03 | 3.59 | 3.22 | 4.17 | 3.59 | 3.22 | 4.17 | 3.59 | 3.22 | 4.17 |
| 6.33 | 3.85 | 3.71 | 4.40 | 3.85 | 3.71 | 4.40 | 3.85 | 3.71 | 4.40 |
| 6.63 | 3.97 | 3.84 | 4.40 | 3.97 | 3.84 | 4.40 | 3.97 | 3.84 | 4.40 |
| 6.93 | 4.14 | 3.90 | 4.33 | 4.14 | 3.90 | 4.33 | 4.14 | 3.90 | 4.33 |
| 7.23 | 4.15 | 3.95 | 4.51 | 4.15 | 3.95 | 4.51 | 4.15 | 3.95 | 4.51 |
| 7.53 | 4.08 | 3.87 | 4.77 | 4.08 | 3.87 | 4.77 | 4.08 | 3.87 | 4.77 |
| 7.83 | 3.92 | 3.92 | 4.52 | 3.92 | 3.92 | 4.52 | 3.92 | 3.92 | 4.52 |
| 8.13 | 3.94 | 3.72 | 4.56 | 3.94 | 3.72 | 4.56 | 3.94 | 3.72 | 4.56 |
| 8.44 | 3.70 | 3.84 | 4.55 | 3.70 | 3.84 | 4.55 | 3.70 | 3.84 | 4.55 |
| 8.74 | 3.32 | 3.49 | 4.59 | 3.32 | 3.49 | 4.59 | 3.32 | 3.49 | 4.59 |
| 9.04 | 3.66 | 3.70 | 4.63 | 3.66 | 3.70 | 4.63 | 3.66 | 3.70 | 4.63 |
| 9.34 | 3.81 | 3.31 | 4.58 | 3.81 | 3.31 | 4.58 | 3.81 | 3.31 | 4.58 |
| 9.64 | 3.76 | 3.18 | 4.47 | 3.76 | 3.18 | 4.47 | 3.76 | 3.18 | 4.47 |
| 9.94 | 3.45 | 3.08 | 3.98 | 3.45 | 3.08 | 3.98 | 3.45 | 3.08 | 3.98 |
| 10.24 | 3.21 | 3.54 | 3.81 | 3.21 | 3.54 | 3.81 | 3.21 | 3.54 | 3.81 |
| 10.54 | 3.26 | 3.24 | 3.74 | 3.26 | 3.24 | 3.74 | 3.26 | 3.24 | 3.74 |
| 10.85 | 3.17 | 3.17 | 3.76 | 3.17 | 3.17 | 3.76 | 3.17 | 3.17 | 3.76 |
| 11.15 | 3.15 | 3.70 | 3.65 | 3.15 | 3.70 | 3.65 | 3.15 | 3.70 | 3.65 |
| 11.45 | 3.20 | 3.44 | 4.09 | 3.20 | 3.44 | 4.09 | 3.20 | 3.44 | 4.09 |
| 11.75 | 3.22 | 3.29 | 4.06 | 3.22 | 3.29 | 4.06 | 3.22 | 3.29 | 4.06 |
| 12.05 | 3.21 | 3.47 | 3.96 | 3.21 | 3.47 | 3.96 | 3.21 | 3.47 | 3.96 |
| 12.35 | 3.27 | 3.05 | 4.17 | 3.27 | 3.05 | 4.17 | 3.27 | 3.05 | 4.17 |
| 12.65 | 3.29 | 2.73 | 4.04 | 3.29 | 2.73 | 4.04 | 3.29 | 2.73 | 4.04 |
| 12.96 | 3.21 | 2.56 | 4.08 | 3.21 | 2.56 | 4.08 | 3.21 | 2.56 | 4.08 |
| 13.26 | 3.09 | 2.65 | 3.97 | 3.09 | 2.65 | 3.97 | 3.09 | 2.65 | 3.97 |
| 13.56 | 3.09 | 2.94 | 4.07 | 3.09 | 2.94 | 4.07 | 3.09 | 2.94 | 4.07 |
| 13.86 | 3.06 | 2.80 | 4.05 | 3.06 | 2.80 | 4.05 | 3.06 | 2.80 | 4.05 |
| 14.16 | 2.87 | 2.63 | 3.80 | 2.87 | 2.63 | 3.80 | 2.87 | 2.63 | 3.80 |
| 14.46 | 2.93 | 2.64 | 4.15 | 2.93 | 2.64 | 4.15 | 2.93 | 2.64 | 4.15 |
| 14.76 | 2.98 | 2.68 | 4.23 | 2.98 | 2.68 | 4.23 | 2.98 | 2.68 | 4.23 |
| 15.06 | 3.18 | 3.14 | 3.89 | 3.18 | 3.14 | 3.89 | 3.18 | 3.14 | 3.89 |
| 15.37 | 2.98 | 3.28 | 3.95 | 2.98 | 3.28 | 3.95 | 2.98 | 3.28 | 3.95 |
| 15.67 | 3.08 | 3.14 | 3.92 | 3.08 | 3.14 | 3.92 | 3.08 | 3.14 | 3.92 |
| 15.97 | 3.37 | 3.25 | 4.02 | 3.37 | 3.25 | 4.02 | 3.37 | 3.25 | 4.02 |
| 16.27 | 3.55 | 3.40 | 4.31 | 3.55 | 3.40 | 4.31 | 3.55 | 3.40 | 4.31 |
| 16.57 | 3.80 | 3.11 | 4.24 | 3.80 | 3.11 | 4.24 | 3.80 | 3.11 | 4.24 |
| 16.87 | 3.86 | 3.03 | 4.23 | 3.86 | 3.03 | 4.23 | 3.86 | 3.03 | 4.23 |
| 17.17 | 3.35 | 3.42 | 4.27 | 3.35 | 3.42 | 4.27 | 3.35 | 3.42 | 4.27 |
| 17.47 | 3.17 | 3.45 | 4.40 | 3.17 | 3.45 | 4.40 | 3.17 | 3.45 | 4.40 |
| 17.78 | 3.01 | 3.30 | 4.09 | 3.01 | 3.30 | 4.09 | 3.01 | 3.30 | 4.09 |
| 18.08 | 2.94 | 3.28 | 4.20 | 2.94 | 3.28 | 4.20 | 2.94 | 3.28 | 4.20 |
| 18.38 | 2.76 | 3.28 | 4.01 | 2.76 | 3.28 | 4.01 | 2.76 | 3.28 | 4.01 |
| 18.68 | 2.79 | 3.39 | 4.08 | 2.79 | 3.39 | 4.08 | 2.79 | 3.39 | 4.08 |
| 18.98 | 2.77 | 3.12 | 4.02 | 2.77 | 3.12 | 4.02 | 2.77 | 3.12 | 4.02 |
| 19.28 | 2.69 | 2.80 | 3.73 | 2.69 | 2.80 | 3.73 | 2.69 | 2.80 | 3.73 |
| 19.58 | 3.07 | 2.64 | 3.69 | 3.07 | 2.64 | 3.69 | 3.07 | 2.64 | 3.69 |
| 19.88 | 2.91 | 2.68 | 3.45 | 2.91 | 2.68 | 3.45 | 2.91 | 2.68 | 3.45 |
| 20.19 | 2.76 | 2.83 | 3.41 | 2.76 | 2.83 | 3.41 | 2.76 | 2.83 | 3.41 |
| 20.49 | 2.64 | 3.15 | 3.54 | 2.64 | 3.15 | 3.54 | 2.64 | 3.15 | 3.54 |
| 20.79 | 2.50 | 3.12 | 3.46 | 2.50 | 3.12 | 3.46 | 2.50 | 3.12 | 3.46 |
| 21.09 | 2.50 | 2.92 | 3.55 | 2.50 | 2.92 | 3.55 | 2.50 | 2.92 | 3.55 |
| 21.39 | 2.79 | 2.73 | 3.76 | 2.79 | 2.73 | 3.76 | 2.79 | 2.73 | 3.76 |
| 21.69 | 2.99 | 2.94 | 3.55 | 2.99 | 2.94 | 3.55 | 2.99 | 2.94 | 3.55 |
| 21.99 | 3.18 | 2.76 | 3.51 | 3.18 | 2.76 | 3.51 | 3.18 | 2.76 | 3.51 |
| 22.29 | 3.02 | 2.69 | 3.37 | 3.02 | 2.69 | 3.37 | 3.02 | 2.69 | 3.37 |
| 22.60 | 2.99 | 2.87 | 3.36 | 2.99 | 2.87 | 3.36 | 2.99 | 2.87 | 3.36 |
| 22.90 | 2.93 | 3.41 | 3.26 | 2.93 | 3.41 | 3.26 | 2.93 | 3.41 | 3.26 |
| 23.20 | 2.88 | 3.26 | 3.43 | 2.88 | 3.26 | 3.43 | 2.88 | 3.26 | 3.43 |
| 23.50 | 2.94 | 3.03 | 3.43 | 2.94 | 3.03 | 3.43 | 2.94 | 3.03 | 3.43 |

### Thermic Response to Food (Days 15-17)

Energy expenditure consists of basal metabolic rate, exercise-induced energy expenditure, and non-exercise thermogenesis. The latter is the most likely area to be affected by anti-obesity agents. Non-exercise thermogenesis includes the thermic response to food, which in rodents can be measured by examining energy expenditure in response to a meal. This was measured after an oral dose of complan (see methods).

The data shows that rimonabant is without effect, whereas both the 3 mg kg⁻¹ and 30 mg kg⁻¹ doses of THCV-BDS have an affect over 2 h as does pure THCV as is shown in Table 2 and FIGS. 2 a) to e).

TABLE 2
Thermic response to food

| | | | | | | | | | | botanical THCV | | |
|---|---|---|---|---|---|---|---|---|---|---|---|---|
| | | | | rimonabant | | | AM 251 | | | (0.3 mg · kg−1 | | |
| Hour | Control | | | (10 mg · kg−1 p.o.) | | | (10 mg · kg−1 p.o.) | | | p.o.) | | |
| 0.22 | 0.84 | 0.41 | 0.98 | 0.67 | 0.41 | 1.08 | 0.45 | 0.50 | 1.00 | 0.50 | 0.59 | 1.25 |
| 0.44 | 1.45 | 0.79 | 1.49 | 1.11 | 1.21 | 2.21 | 1.11 | 1.31 | 1.97 | 1.46 | 1.36 | 1.98 |
| 0.67 | 1.63 | 1.34 | 1.82 | 1.38 | 1.56 | 2.67 | 1.81 | 1.97 | 2.49 | 1.81 | 1.56 | 2.50 |
| 0.89 | 1.85 | 1.59 | 1.69 | 1.43 | 1.86 | 2.87 | 2.19 | 2.38 | 2.78 | 2.20 | 1.76 | 2.53 |
| 1.11 | 1.89 | 1.91 | 1.91 | 1.51 | 2.14 | 3.26 | 2.52 | 2.78 | 3.40 | 2.36 | 1.91 | 2.61 |
| 1.33 | 2.08 | 2.00 | 2.26 | 1.48 | 2.32 | 3.28 | 2.86 | 2.85 | 3.64 | 2.53 | 2.00 | 2.78 |
| 1.56 | 2.35 | 2.31 | 2.19 | 1.53 | 2.63 | 3.20 | 3.05 | 3.14 | 3.53 | 2.81 | 2.15 | 2.86 |
| 1.78 | 2.71 | 2.34 | 2.05 | 1.54 | 2.67 | 3.11 | 3.30 | 3.25 | 3.50 | 2.73 | 2.17 | 2.88 |
| 2.00 | 2.73 | 2.46 | 2.04 | 1.36 | 2.63 | 2.97 | 3.30 | 3.44 | 3.44 | 2.72 | 2.28 | 2.80 |
| 2.22 | 2.61 | 2.41 | 1.98 | 1.33 | 2.91 | 2.98 | 3.30 | 4.10 | 3.46 | 2.81 | 2.53 | 2.64 |
| 2.44 | 2.44 | 2.66 | 2.12 | 1.42 | 2.74 | 2.96 | 3.38 | 4.00 | 3.68 | 2.97 | 2.41 | 2.80 |
| 2.67 | 2.40 | 2.99 | 2.25 | 1.40 | 2.83 | 2.91 | 3.42 | 4.24 | 3.78 | 2.82 | 2.38 | 3.27 |
| 2.89 | 2.56 | 3.01 | 2.53 | 1.30 | 2.75 | 3.07 | 3.56 | 4.26 | 3.99 | 3.09 | 2.59 | 3.42 |
| 3.11 | 2.42 | 3.24 | 2.72 | 1.20 | 2.82 | 3.09 | 3.69 | 4.26 | 3.78 | 3.43 | 2.99 | 3.37 |
| 3.33 | 2.28 | 3.51 | 2.87 | 1.28 | 2.74 | 3.22 | 3.63 | 4.23 | 3.80 | 3.31 | 3.01 | 3.21 |
| 3.56 | 2.43 | 3.49 | 2.85 | 1.50 | 2.83 | 3.25 | 3.79 | 4.24 | 3.62 | 3.30 | 2.99 | 2.98 |
| 3.78 | 2.33 | 3.58 | 2.53 | 1.56 | 2.96 | 3.02 | 3.79 | 4.21 | 3.54 | 3.13 | 2.97 | 2.87 |
| 4.00 | 2.52 | 3.66 | 2.52 | 1.56 | 2.90 | 3.09 | 4.05 | 4.24 | 3.79 | 3.27 | 2.99 | 2.98 |

| | botanical THCV | | | botanical THCV | | | pure THCV | | |
|---|---|---|---|---|---|---|---|---|---|
| Hour | (3 mg · kg−1 p.o.) | | | (3 mg · kg−1 p.o.) | | | (0.3 mg · kg−1 p.o.) | | |
| 0.22 | 0.67 | 0.91 | 1.26 | 0.67 | 0.91 | 1.26 | 0.67 | 0.91 | 1.26 |
| 0.44 | 1.78 | 1.70 | 1.98 | 1.78 | 1.70 | 1.98 | 1.78 | 1.70 | 1.98 |
| 0.67 | 2.30 | 2.06 | 2.49 | 2.30 | 2.06 | 2.49 | 2.30 | 2.06 | 2.49 |
| 0.89 | 2.51 | 2.42 | 2.51 | 2.51 | 2.42 | 2.51 | 2.51 | 2.42 | 2.51 |
| 1.11 | 2.51 | 2.74 | 2.64 | 2.51 | 2.74 | 2.64 | 2.51 | 2.74 | 2.64 |
| 1.33 | 2.50 | 2.82 | 2.67 | 2.50 | 2.82 | 2.67 | 2.50 | 2.82 | 2.67 |
| 1.56 | 2.53 | 2.81 | 3.13 | 2.53 | 2.81 | 3.13 | 2.53 | 2.81 | 3.13 |
| 1.78 | 2.54 | 2.76 | 3.31 | 2.54 | 2.76 | 3.31 | 2.54 | 2.76 | 3.31 |
| 2.00 | 2.45 | 2.62 | 3.18 | 2.45 | 2.62 | 3.18 | 2.45 | 2.62 | 3.18 |
| 2.22 | 2.61 | 2.74 | 3.44 | 2.61 | 2.74 | 3.44 | 2.61 | 2.74 | 3.44 |
| 2.44 | 2.71 | 2.74 | 3.18 | 2.71 | 2.74 | 3.18 | 2.71 | 2.74 | 3.18 |
| 2.67 | 2.66 | 2.83 | 3.10 | 2.66 | 2.83 | 3.10 | 2.66 | 2.83 | 3.10 |
| 2.89 | 2.71 | 3.01 | 3.09 | 2.71 | 3.01 | 3.09 | 2.71 | 3.01 | 3.09 |
| 3.11 | 2.68 | 3.16 | 3.02 | 2.68 | 3.16 | 3.02 | 2.68 | 3.16 | 3.02 |
| 3.33 | 2.68 | 3.34 | 3.04 | 2.68 | 3.34 | 3.04 | 2.68 | 3.34 | 3.04 |
| 3.56 | 2.61 | 3.49 | 3.02 | 2.61 | 3.49 | 3.02 | 2.61 | 3.49 | 3.02 |
| 3.78 | 2.43 | 3.48 | 3.03 | 2.43 | 3.48 | 3.03 | 2.43 | 3.48 | 3.03 |
| 4.00 | 2.70 | 3.49 | 3.13 | 2.70 | 3.49 | 3.13 | 2.70 | 3.49 | 3.13 |

### Discussion:

On a dosage basis pure THCV seems to be at least 10-fold and possibly 30-fold more potent than rimonabant on energy expenditure. Both high dose THCV-BDS and pure THCV (0.3 mg kg⁻¹) appeared to be more effective than rimonabant. The effect on energy expenditure was much greater after 10-12 days treatment than at 3-5 days, suggesting some induction of thermogenesis.

In the current study the thermic response to food was determined on days 15-17. Except for rimonabant, all treatments gave an increase but this increase was not sufficient to be responsible for the increase in 24 h energy expenditure.

The treatments resulted in changes in the percentage body fat although these were not statistically significant using analysis of variance. Of particular interest was the finding that pure THCV appeared to be at least as effective as rimonabant at a 30-fold lower dose level without any effect on growth. The fall in leptin levels mirrored the effect on percentage body fat, but using analysis of variance only AM251 gave a significant effect.

### EXAMPLE 2

### The Effect of Cannabidiol in Diabetes, Obesity and Related Metabolic Disorders

Mechoulam and colleagues have shown that cannabidiol treatment significantly reduces the incidence of diabetes in NOD mice, which is a model of type I autoimmune mediated diabetes, from 86% in non-treated controls to 30% in cannabidiol treated mice. The CBD treatment also resulted in a significant reduction in plasma levels of IFN-gamma and TNF-alpha. Histological examination of the pancreatic islets revealed reduced insulinitis.

Inflammation is also a feature of type II diabetic animals and man. Therefore the current study examined whether cannabidiol had a similar effect in a model of type II diabetes, namely the C57B1/Ks db/db mouse. This animal lacks a functional leptin receptor and initially shows gross obesity and insulin resistance. However, as a result of the C57B1/Ks genetic background rather than the C57B1/6, the mice show loss of pancreatic function from around 6/7 weeks of age and by 10 weeks show frank diabetes.

### Methods:

The mice were given chow diet (Bantin and Kingman, no 1 diet) and water ad lib. The mice were kept under controlled lighting conditions (lights on 08.00 h, 12 h light/12 h dark) and at a room temperature of 21°±1° C.

A control
B Pure CBD (5 mg/kg oral)
C Pure CBD (1 mg/kg oral)
D Pure CBD (5 mg/kg i.p.)

### Measurements:

Daily: Food and water intake
Twice weekly: Body weight and glucose
Day 1: Weigh mice, dose compounds, provide measured amount of food, provide measured amount of water
Days 2, 5 and 8: Fed blood glucose concentration
Day 9: Oral glucose tolerance
Days 12 and 16: Fed blood glucose concentration
Day 19: 24 hr glycaemic assay
Days 22, 26 and 29: Fed blood glucose concentration
Day 33: 24 hr glycaemic assay
Day 38: Fed blood glucose concentration
Day 39: Body composition by Dexascan in anaesthetised mice
Day 41: Termination. Take blood samples for glucose, free fatty acids, triglycerides, cholesterol, insulin and HbA

### Key Results:

### Effects of Cannabidiol on Plasma Insulin Concentration

The development of diabetes in the db/db mouse model arises as a result of a combination of insulin resistance and compromised insulin secretion. Thus, diabetes ensues when insufficient insulin is released into the circulation to overcome the resistance. Naturally the diabetes can be overcome by either reducing the insulin resistance or enhancing secretion or a combination of the two.

The effect of treatment on plasma insulin concentration is potentially complex. Thus, improving insulin resistance could reduce the plasma insulin concentration by reducing demand. Improving islet cell function by suppressing islet inflammation might be expected to increase plasma insulin concentrations. In the current study plasma insulin was determined at several times over the course of treatment.

On day 9, insulin was determined in 5 h-fasted mice, prior to the glucose tolerance test. There were no significant effects but both of the 5 mg kg⁻¹ treatments tended to give higher plasma insulin concentrations.

Plasma insulin was also measured during the 24 h glucose profiles and no consistent effect was seen.

### Discussion:

The current experiment did not show significant differences in glycaemic control between mice receiving cannabidiol and control in this mouse model. It is possible that there might be some improvement in islet cell function and mass of cells.

Cannabidiol may therefore prevent the development of diabetes and could be used as a monotherapy and in combination with an insulin sensitizer such as rosiglitazone.

### EXAMPLE 3

The Effect of Tetrahydrocannabivarin and/or Cannabidiol in Diabetes, Obesity and Related Metabolic Disorders

Terahydrocannabivarin (THCV) is a potent antagonist of the cannabinoids WIN55212-2 and anandamide in the mouse isolated vas deferens preparation. Previous studies in DIO mice have suggested that it has some CB₁ antagonist properties and is more potent than the CB-1 antagonists rimonabant and AM251.

Cannabidiol has been reported to have some protective effect on pancreatic islets in the NOD mouse, which is a model of type I diabetes.

Consequently, further studies were initiated on both agents and the combination in the C57B1/6 ob/ob mouse, which is a model of insulin resistance, obesity and the metabolic syndrome.

A chronic dosing, 28-day study was undertaken where ob/ob mice were dosed daily at 09:00 by oral gavage with either:

### A Control

B AM 251 10 mg/kg
C Pure THCV 0.3 mg/kg
D Pure THCV 3.0 mg/kg
E Pure THCV 0.3 mg/kg+CBD BDS containing CBD at 0.3 mg/kg
F Pure THCV 3.0 mg/kg+CBD BDS containing 3.0 mg/kg
G Pure CBD 3.0 mg/kg

Animals were acclimatised during Days 1-2 of the study and dosing was started on Day 3.

Measurements were taken to provide data for the following:
Daily: Food and water intake
Twice weekly: Body weight
Day −1: Commencement of daily food and water measurements at 17:00 throughout study
Day 7: Oral glucose tolerance (glucose load 3 g/kg) in 5 h-fasted mice measuring glucose at −30, 0, 30, 60, 90, 120 min and insulin at −30 min
Day 10: 24 h energy expenditure by indirect calorimetry
Day 17: Thermic response to a mixed meal then fast mice for 2 h dose mice then 60 mins later give a complan meal (orally) measure energy expenditure from dosing for 4 h post complan meal
Day 22: Oral glucose tolerance in 5 h-fasted mice (repeat day 7 study)
Day 28: Body composition by Dexascan in anaesthetised mice; measure nose-anus length; blood sample from fed mice for glucose, lactate, insulin, triglycerides, cholesterol, HDL-cholesterol
Day 35: Fast overnight (17 h fast)
Day 36: Blood sample for glucose, free fatty acids, insulin and adiponectin dose mice and take blood sample 2-3 h post-dosing for drug levels and endocannabinoid plasma levels. Kill mice using schedule 1 method—remove brain for endocannabinoid level. Measure weight of a discrete fat pad. Freeze clamp liver, weigh and take samples for measurement of liver lipid and glycogen

### Key Results:

### Effects of Treatment on Energy Expenditure and Respiratory Quotient

The mice showed the normal diurnal pattern of energy expenditure with total energy expenditure being significantly greater during the early dark phase of the light cycle than during the light phase. All treatments increased 24 h energy expenditure when measured on day 10, with the exception of cannabidiol given alone. Moreover, the diurnal pattern was similar to controls for each treatment.

To gain further insight into the effects of these cannabinoids on energy expenditure, the thermic response to food was assessed using oral dosage of a Complan® meal. All treatments increased the post-prandial energy expenditure. The response pattern was similar to the 24 h pattern, with AM251 (10 mg kg⁻¹), pure THCV (3 mg kg⁻¹) and both the low dose and high dose combination having a significant effect (Table 3, FIG. 3).

TABLE 3
Thermic effect of a meal: 3-hour energy
expenditure per animal

| | | | | | | | | THCV + CBD | | THCV + CBD | | | |
|---|---|---|---|---|---|---|---|---|---|---|---|---|---|
| | | AM251 | | THCV | | THCV | | BDS both | | BDS both | | CBD | |
| Control | | 10 mg/kg | | 0.3 mg/kg | | 3.0 mg/kg | | 0.3 mg/kg | | 3.0 mg/kg | | 3.0 mg/kg | |
| 6.20 | 5.91 | 9.00 | 9.38 | 8.18 | 7.04 | 8.94 | 8.79 | 10.20 | 9.45 | 12.03 | 10.28 | 7.84 | 8.34 |

Interestingly, although pure CED did not significantly increase energy expenditure relative to controls, the botanical drug substance CBD did appear to enhance the energy expenditure effect of THCV.

Effects on plasma analytes in ad-lib fed mice

The effects on cholesterol and HDL-cholesterol provide the most surprising results. Pure CBD reduced the total plasma cholesterol (Table 4, FIG. 4) whilst at the same time increasing the plasma HDL-cholesterol concentration (Table 5, FIG. 5) so that the HDL-cholesterol as a percentage of total cholesterol rose from 40% to greater than 80% (Table 6, FIG. 6).

The combination of THCV-CBD BDS also significantly increased the HDL-cholesterol concentration but only the high dose combination tended to reduce total cholesterol and thereby impact on the ratio.

TABLE 4
Fed plasma cholesterol day 28

| | | | | THCV + | THCV + | |
|---|---|---|---|---|---|---|
| | AM 251 | THCV | THCV | CBD both | CBD both | CBD |
| control | 10 mg · kg⁻¹ | 0.3 mg · kg⁻¹ | 3.0 mg · kg⁻¹ | 0.3 mg · kg⁻¹ | 3.0 mg · kg⁻¹ | 3.0 mg · kg⁻¹ |
| 4.94 | 6.01 | 6.45 | 6.46 | 9.45 | 5.42 | 2.90 |
| 4.55 | 6.43 | 5.93 | 7.66 | 9.49 | 5.12 | 5.38 |
| 5.24 | 5.75 | 5.62 | 7.05 | 8.11 | 4.21 | 3.50 |
| 4.04 | 5.82 | 7.12 | 6.30 | 7.05 | 5.02 | 3.64 |
| 6.27 | 7.85 | 8.60 | 6.94 | 7.29 | 3.23 | 4.22 |
| 5.99 | 6.66 | 6.43 | 6.59 | 7.71 | 5.38 | 3.77 |
| 7.62 | 6.15 | 5.99 | 6.20 | 5.33 | 4.18 | 5.02 |
| 6.24 | — | 6.21 | 7.22 | 5.86 | 3.72 | — |

TABLE 5
Fed HDL cholesterol day 28

| | | | | THCV + | THCV + | |
|---|---|---|---|---|---|---|
| | AM 251 | THCV | THCV | CBD both | CBD both | CBD |
| control | 10 mg · kg⁻¹ | 0.3 mg · kg⁻¹ | 3.0 mg · kg⁻¹ | 0.3 mg · kg⁻¹ | 3.0 mg · kg⁻¹ | 3.0 mg · kg⁻¹ |
| 2.07 | 1.97 | 2.21 | 2.94 | 3.43 | 3.37 | 2.98 |
| 2.03 | 2.24 | 2.25 | 2.57 | 3.19 | 3.02 | 3.38 |
| 2.14 | 2.00 | 2.14 | 2.28 | 3.03 | 2.90 | 3.18 |
| 1.88 | 2.03 | 2.16 | 2.17 | 3.06 | 3.09 | 3.31 |
| 2.06 | 2.26 | 2.16 | 2.16 | 3.00 | 2.87 | 3.00 |
| 2.10 | 2.04 | 2.16 | 2.24 | 3.17 | 3.22 | 3.36 |
| 2.18 | 2.19 | 2.35 | 2.51 | 3.16 | 3.17 | 3.63 |
| 2.36 | — | 2.33 | 2.98 | 3.65 | 3.17 | — |

TABLE 6
percentage HDL cholesterol day 28

| | | | | THCV + | THCV + | |
|---|---|---|---|---|---|---|
| | AM 251 | THCV | THCV | CBD both | CBD both | CBD |
| control | 10 mg · kg⁻¹ | 0.3 mg · kg⁻¹ | 3.0 mg · kg⁻¹ | 0.3 mg · kg⁻¹ | 3.0 mg · kg⁻¹ | 3.0 mg · kg⁻¹ |
| 41.9 | 32.8 | 34.3 | 45.6 | 36.3 | 62.1 | 102.9 |
| 44.6 | 34.9 | 37.9 | 33.6 | 33.6 | 58.8 | 62.7 |
| 40.8 | 34.8 | 38.0 | 32.4 | 37.3 | 68.9 | 90.8 |
| 46.6 | 34.8 | 30.4 | 34.5 | 43.4 | 61.6 | 90.9 |
| 32.8 | 28.8 | 25.1 | 31.1 | 41.1 | 88.8 | 71.1 |
| 35.0 | 30.6 | 33.6 | 34.0 | 41.1 | 59.7 | 88.9 |
| 28.6 | 35.6 | 39.3 | 40.4 | 59.3 | 75.9 | 72.3 |
| 37.8 | — | 37.5 | 41.3 | 62.3 | 85.2 | — |

Plasma insulin was not affected by treatment.

### Effect on Liver Glycogen and Triglycerides

The liver triglyceride content was markedly affected by the treatments. CBD, both alone and in combination with THCV reduced liver triglyceride content markedly. In contrast liver triglyceride content was increased by AM251 and low dose pure THCV (Table 7, FIG. 7).

TABLE 7
Liver triglycerides concentration

| | | | | THCV + | THCV + | |
|---|---|---|---|---|---|---|
| | AM 251 | THCV | THCV | CBD both | CBD both | CBD |
| control | 10 mg · kg⁻¹ | 0.3 mg · kg⁻¹ | 3.0 mg · kg⁻¹ | 0.3 mg · kg⁻¹ | 3.0 mg · kg⁻¹ | 3.0 mg · kg⁻¹ |
| 9.68 | 10.36 | 16.72 | 13.92 | 9.44 | 6.13 | 5.47 |
| 8.04 | 13.65 | 9.33 | 18.27 | 8.50 | 5.54 | 4.78 |
| 9.21 | 17.44 | 14.97 | 14.09 | 5.92 | 6.50 | 5.46 |
| 5.87 | 13.87 | 17.16 | 8.70 | 7.04 | 4.14 | 4.02 |
| 11.11 | 16.47 | 11.16 | 15.20 | 5.69 | 3.78 | 4.64 |
| 12.53 | 17.40 | 14.94 | 10.77 | 6.45 | 4.51 | 4.58 |
| 10.69 | 11.05 | 15.48 | 9.78 | 7.62 | 3.20 | 5.42 |
| 14.35 | — | 9.10 | 12.06 | 4.10 | 3.30 | — |

### Discussion:

The most striking finding of this study came from the cannabinoid CBD. Given on its own it reduced the total plasma cholesterol concentration whilst increasing the amount as well as the percentage of HDL-cholesterol. It had no effect on plasma triglycerides but reduced the hepatic triglycerides.

CBD appears to have utility as an agent that increases HDL-cholesterol whilst lowering total cholesterol and liver lipids.

The effect of the low dose combination of THCV and CBD on HDL-cholesterol together with the lack of effect shown by pure THCV would suggests that a lower dose of CBD would raise HDL-cholesterol but might not lower total cholesterol.

The effect of THCV alone and in combination with cannabidiol BDS on energy expenditure was very surprising. The increase in 24 h energy expenditure and the thermic response to food was remarkable.

Why the combination of cannabinoids appeared to have a greater effect on energy expenditure than either THCV or CBD alone is not known. It could be that there is a synergistic effect between THCV and CBD.

### EXAMPLE 4

The example described below investigated whether the cannabinoids, cannabidiol (CBD) and tetrahydrocannabivarin (THCV), act via the peroxisome proliferator-activated receptor gamma (PPARγ), which is known to be activated by Δ⁹-tetrahydrocannabinol.

Agonists of the PPARγ isoform improve insulin sensitivity and are often used in the management of type II diabetes. Additionally, PPARγ agonists have been shown to have positive cardiovascular effects, which include in vitro evidence of increased availability of nitric oxide (NO), and in vivo reductions in blood pressure and attenuation of atherosclerosis.

Some of the beneficial effects of PPARγ ligands are brought about by the anti-inflammatory actions of PPARγ cytokines, increasing anti-inflammatory cytokines, and inhibition of inducible nitric oxide synthase (iNOS) expression. It is therefore thought that the use of PPARγ ligands might be a useful treatment option in the pharmaceutical management of metabolic syndrome or diseases and conditions associated with an increased risk of metabolic syndrome.

In vitro vascular studies were carried out in rat isolated aortae by wire myography. PPARγ activation was investigated using reporter gene assays, a PPARγ competition-binding assay and an adipogenesis assay.

Both THCV and CBD were dissolved in ethanol to a stock concentration of 10 mM and further dilutions were made using distilled water.

### Results:

### Time-dependent Effects of CBD and THCV in the Aorta

CBD (10 μM) caused significant time-dependent relaxation of the rat aorta compared to vehicle control at all time-points over the course of 2 h (2 h, vehicle 19.7±2.4% cf CBD 69.7±4.0% relaxation, n=13, P<0.001. After 2 h, the residual relaxation (the vasorelaxant effect of CBD minus the vasorelaxant effect of vehicle and time) was 50.1±3.3% relaxation.

CBD had no effect on basal tension over time (2 h, vehicle −0.02±0.01 g cf CBD −0.03±0.01 g, n=7).

In pre-contracted aortae, THCV (10 μM) had no effect on tone until after 105 minutes, and after 120 min, vasorelaxation to THCV was 28.7±4.6% relaxation (n=10), compared to 15.1±4.6% (P<0.01) in control arteries.

In the presence of the PPARγ receptor antagonist GW9662 (1 μM), the residual vasorelaxant effect of CBD was significantly reduced after 1 h of incubation. The vasorelaxant effect of CBD was similar in endothelium-denuded and control aortae. Similarly, in the presence of the nitric oxide synthase inhibitor, L-NAME (300 μM), the residual vasorelaxant effect of CBD was not different to that observed in control conditions.

The CB₁ receptor antagonist AM251 (1 μM) did not significantly affect the time-dependent vascular responses to CBD. The CB₂ receptor antagonist SR144528 (1 μM) significantly inhibited the residual vasorelaxant effects of CBD between 45 min to 90 min. Pre-treating arteries with either PTX (200 ng ml⁻¹, 2 h) or with capsaicin (10 μM, 1 h) had no effect on the vascular response to CBD over time.

When arteries were contracted with a high potassium buffer, there was no difference in the vasorelaxant effect of CBD compared with control. By contrast, in vessels where tone was induced with U46619 in calcium free buffer, the vasorelaxant effect of CBD was significantly blunted compared with control.

The potency and maximal contractile response to the re-introduction of calcium in calcium free, high potassium Krebs-Hensleit solution was significantly reduced in a concentration-dependent manner the presence of CBD from 1 μM to 30 μM. The calcium channel blocker, verapamil, caused significant vasorelaxation of pre-constricted vessels as CBD, although with a more rapid onset.

Effects of Chronic Treatment of Rats with CBD on Vascular Responses in Isolated Arteries

Animals were treated for 2 weeks with either vehicle or CBD, and investigations of arterial function made.

In small resistance mesenteric vessels, the maximal contractile responses to methoxamine were significantly lower in CBD-treated animals than in vehicle-treated animals (Rₘₐₓ 1.56±0.13 g vs CBD 2.20±0.13 g increase tension, n=7, P<0.001). CBD treatment caused an additional decrease in the potency of methoxamine (pEC₅₀ veh 5.94±0.08 vs CBD 5.79±0.10, P<0.05).

The maximal response to methoxamine in the aorta was also significantly higher in vehicle-treated animals (2.32±0.20 g increase tension, n=6) compared to CBD-treated animals (1.63±0.21 g increase tension, n=7, P<0.001).

Repeated treatment with CBD did not affect the vasorelaxant responses to acetylcholine in small resistance mesenteric arteries. However, in the aorta, CBD treatment significantly decreased the potency of acetylcholine (pEC₅₀ control 6.17±0.31 vs CBD-treated 5.37±0.40, n=6, P<0.01).

### Transcriptional Transactivation Assays

To determine whether CBD stimulates PPARγ, transactivation assays were performed in homologous cells transiently overexpressing PPARγ and RXRα in combination with a luciferase reporter gene (3×PPRE TK luc).

In these assays, the synthetic PPARγ agonist rosiglitazone (10 μM) significantly stimulated the transcriptional activity of PPARγ compared to vehicle-treated cells transfected with all DNA (148±7 cf 319±7 relative luciferase activity (per ng ml⁻¹ protein), P<0.01).

Likewise, CBD also significantly stimulated the transcriptional activity of PPARγ compared to untreated-cells at 10 μM (305±18 relative luciferase activity, P<0.01) and 20 μM (470±37 relative luciferase activity, P<0.01) in a concentration-dependent manner.

THCV had no effect on PPARγ transcriptional activity at any concentration tested.

### Induction of Adipocyte Differentiation

3T3L1 cells were cultured until confluent and then treated for 8 days with either CBD or rosiglitazone. Cells were fixed and stained with Oil red ◯ to identify fat droplets, to the presence of which indicates differentiation of fibroblasts into adipocytes. Untreated cells showed some signs of differentiation, but the majority of cells retained their spindle shape with little Oil Red O staining. Rosiglitazone induced differentiation of 3T3 L1 cells to adipocytes, as evidenced by large amounts of Oil Red O staining indicating fat droplet accumulation within the cytoplasm. In the presence of CBD, signs of fat droplet accumulation were apparent at all concentrations tested in a concentration-dependent manner.

### CONCLUSIONS

These data provide strong evidence that CBD is a PPARγ agonist, and suggest a novel means by which the effects of CBD could be brought about. In light of the emerging evidence that PPARγ ligands have beneficial effects in type II diabetes, the cardiovascular system and potentially in a wide variety of other disorders including cancer, gastroinflammatory disorders and many skin diseases, these data provide evidence that CBD and potentially CBD in combination with THCV could be useful in the prevention or treatment of diabetes, obesity and related metabolic disorders.

1-35. (canceled)

36. A method of controlling cholesterol levels in a subject, comprising administering to a subject in need thereof an effective amount of cannabidiol (CBD) alone or in combination with another cannabinoid.

37. A method of increasing energy expenditure in a subject, comprising administering to a subject in need thereof an effective amount of tetrahydrocannabivarin (THCV) alone or in combination with another cannabinoid.

38. The method as claimed in claim 36, wherein total plasma cholesterol is reduced.

39. The method as claimed in claim 36, wherein the percentage of HDL cholesterol relative to total cholesterol is increased.

40. The method as claimed in claim 36, wherein the other cannabinoid is tetrahydrocannabivarin (THCV).

41. The method as claimed in claim 36, wherein the CBD is in the form of a cannabinoid-containing plant extract derived from at least one cannabis plant.

42. The method as claimed in claim 41, wherein the cannabinoid-containing plant extract from at least one cannabis plant is a botanical drug substance.

43. The method as claimed in claim 41, wherein the cannabinoid-containing plant extract from at least one cannabis plant comprises all or some of the naturally occurring cannabinoids present in the plant.

44. The method as claimed in claim 43, wherein all or a significant amount of any tetrahydrocannabinol (THC) occurring in the cannabis-containing plant extract has been removed.

45. The method as claimed in claim 36, wherein the CBD or any other cannabinoid is/are in a substantially pure or isolated form.

46. The method as claimed in claim 36, wherein the CBD or any other cannabinoid is/are in a synthetic form.

47. The method as claimed in claim 38, wherein the CBD is present in a dose effective to bring about a reduction in total plasma cholesterol.

48. The method as claimed in claim 47, wherein the effective dose of CBD is between 0.1 mg/kg and 5.0 mg/kg.

49. The method as claimed in claim 48 wherein the other cannabinoid is tetrahydrocannabivarin (THCV), and wherein the THCV is present in an amount of between 0.3 mg/kg and 30.0 mg/kg.

50. The method as claimed in claim 36, wherein the cholesterol levels are controlled as part of a regime to manage or treat type I or type II diabetes, obesity, dyslipidaemia (including atherogenic dyslipidaemia), related metabolic disorders and cardiovascular disease.

51. The method as claimed in claim 40, wherein the CBD and THCV are in a predefined ratio by weight.

52. The method as claimed in claim 36, further comprising administering one or more other drugs used in the treatment of diabetes, obesity, dyslipidaemia (including atherogenic dyslipidaemia), related metabolic disorders or cardiovascular disease.

53. The method as claimed in claim 52, wherein the one or more other drugs is either a drug to reduce the insulin resistance or enhance secretion or a combination of the two.
